(19) United States
(12) Patent Application Publication
(10) Pub. No.: US 2026/0250316 A1
(43) Pub. Date: Aug. 27, 2026

(54) CRYSTALLINE FORMS OF A MACROCYCLIC PEPTIDE ANTIBIOTIC

(71) Applicant: Hoffmann-La Roche Inc., Little Falls, NJ (US)

(72) Inventors: Ralph Diodone, Breisach (DE); André Egli, Basel (CH); Dennis Dimo Enkelmann, Freiburg im Breisgau (DE); Michel Dominik Lindemer, Basel (CH); Valentin Andreas Stadelmann, Bettingen (CH)

(73) Assignee: Hoffmann-La Roche Inc., Little Falls, NJ (US)

(21) Appl. No.: 18/854,508

(22) PCT Filed: Apr. 5, 2023
(86) PCT No.: PCT/EP2023/058906

§ 371 Date: Oct. 4, 2024

(30) Foreign Application Priority Data
Apr. 7, 2022 (EP) 22167057.3

(51) Int. Cl. C07K 5/09; A61K 38/00; A61K 47/02

(52) U.S. Cl. CPC C07K 5/0815; A61K 47/02; A61K 38/00

(57) ABSTRACT

Described herein are the mono hydrochloric acid salt, as well as the free zwitterion of the macrocyclic peptide antibiotic 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid. Also described are amorphous and crystalline forms of said hydrochloric acid salt and free zwitterion, as well as methods of making the same and methods of using the same in medical therapy.

### FIELD OF THE INVENTION

This invention relates to the novel mono hydrochloric acid salt (hereinafter referred to as “HCl salt”), as well as to the novel free zwitterion (hereinafter referred to as “free base”) of the macrocyclic peptide antibiotic 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid. The invention also relates to the amorphous form and certain crystalline forms of said HCl salt and free base. Finally, the invention relates to pharmaceutical compositions comprising said novel forms, to processes for making them and to their use in medical therapy.

### BACKGROUND OF THE INVENTION

WO2019206853, the entire contents of which are incorporated herein by reference, discloses the peptide macrocycle 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula Ia), which has potent antibacterial properties.

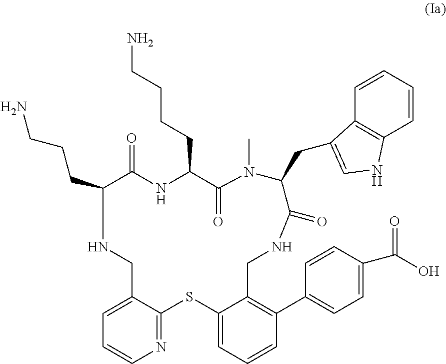 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

More particularly, WO2019206853 discloses a tetrakis tetrafluoroacetic acid (TFA) salt of the compound of Formula (Ia). However, due to toxicity of TFA salts, said TFA salt cannot be employed directly as an active pharmaceutical ingredient for the treatment of bacterial infections.

On the other hand, the “free base” (i.e., free zwitterion) of the compound of Formula (Ia) was found to be hardly soluble in water, which is not acceptable for a pharmaceutical compound that is foreseen to be administered intravenously.

In addition, it was found that the API contained high levels of palladium and sodium chloride impurities from the manufacturing process, which made tedious purifications steps necessary (e.g., nanofiltration to remove sodium chloride). However, repeated purification is highly undesirable in the large scale manufacture of pharmaceutical drugs.

In view of the above, there is an unmet need for new forms of the compound of formula (Ia), which ultimately allow it to be employed as a drug for the treatment of bacterial infections.

### SUMMARY OF THE INVENTION

It has now surprisingly been found that the HCl salt described herein displays a dramatically increased solubility compared to the free base, especially in aqueous media, which is of utmost importance for intravenous administration.

Furthermore, it has surprisingly been found that a certain crystalline form of the free base greatly facilitates the purification of the drug substance, allowing manufacture on an industrial scale under GMP conditions.

Thus, in a first aspect, the present invention provides a mono hydrochloric acid salt of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula I).

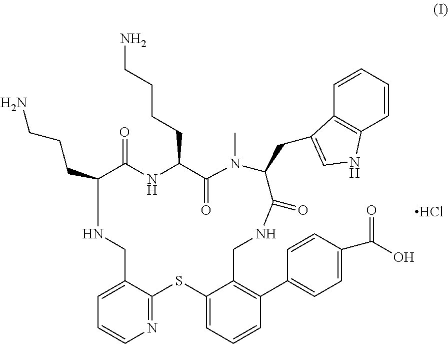 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12 ; Cl]

In a further aspect, the present invention provides certain crystalline forms and an amorphous form of the mono hydrochloric acid salt of Formula (I) as described herein, as well as processes for making the same, methods of using the same and pharmaceutical compositions comprising the same.

In a further aspect, the present invention provides the free base, i.e., free zwitterion, of 4-[(11 S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula Ia).

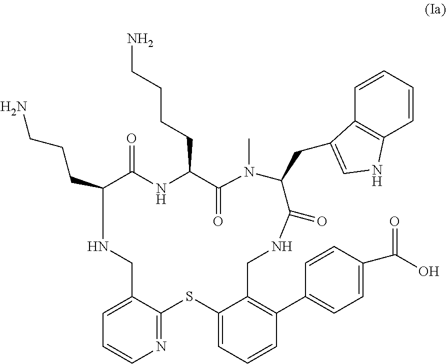 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

In a further aspect, the present invention provides certain crystalline forms and an amorphous form of the free base of formula (Ia) as described herein, as well as processes for making the same, methods of using the same and pharmaceutical compositions comprising the same.

### BRIEF DESCRIPTION OF THE FIGURES

FIG. 1 shows a characteristic XRPD diffraction pattern of the crystalline polymorph form A1 of the mono hydrochloric acid salt of formula (I) described in Example 3.

FIG. 2 shows a characteristic FT Raman spectrum of the crystalline polymorph form A1 of the mono hydrochloric acid salt of formula (I) described in Example 3.

FIG. 3 shows a characteristic ATR-FTIR spectrum of the crystalline polymorph form A1 of the mono hydrochloric acid salt of formula (I) described in Example 3.

FIG. 4 shows a characteristic XRPD diffraction pattern of the crystalline polymorph form A2 of the mono hydrochloric acid salt of formula (I) described in Example 4.

FIG. 5 shows a characteristic FT Raman spectrum of the crystalline polymorph form A2 of the mono hydrochloric acid salt of formula (I) described in Example 4.

FIG. 6 shows a characteristic ATR-FTIR spectrum of the crystalline polymorph form A2 of the mono hydrochloric acid salt of formula (I) described in Example 4.

FIG. 7 shows a characteristic XRPD diffraction pattern of the crystalline polymorph form “Pattern 4” of the mono hydrochloric acid salt of formula (I) described in Example 5.

FIG. 8 shows a characteristic XRPD diffraction pattern of the crystalline polymorph form “Pattern 5” of the mono hydrochloric acid salt of formula (I) described in Example 6.

FIG. 9 shows a characteristic FT Raman spectrum of the amorphous form of the mono hydrochloric acid salt of formula (I) described in Example 2 and of the amorphous form of the free base of formula (Ia) described in Example 7.

FIG. 10 shows a characteristic ATR-FTIR spectrum of the amorphous form of the mono hydrochloric acid salt of formula (I) described in Example 2.

FIG. 11 shows a characteristic XRPD diffraction pattern of the crystalline polymorph form A1 of the free base of formula (Ia) described in Example 8.

FIG. 12 shows a characteristic FT Raman spectrum of the crystalline polymorph form A1 of the free base of formula (Ia) described in Example 8.

FIG. 13 shows a characteristic ATR-FTIR spectrum of the crystalline polymorph form A1 of the free base of formula (Ia) described in Example 8.

FIG. 14 shows a characteristic XRPD diffraction pattern of the crystalline polymorph Form B of the free base of formula (Ia) described in Examples 9 and 10.

FIG. 15 shows a characteristic FT Raman spectrum of the crystalline polymorph Form B of the free base of formula (Ia) described in Examples 9 and 10.

FIG. 16 shows a characteristic ATR-FTIR spectrum of the crystalline polymorph Form B of the free base of formula (Ia) described in Examples 9 and 10.

FIG. 17 shows a characteristic ATR-FTIR spectrum of the amorphous form of the free base of formula (Ia) described in Example 7.

FIG. 18 shows a characteristic XRPD diffraction pattern of the crystalline polymorph form “Pattern 10.1” of the free base of formula (Ia) described in Example 11.

### DETAILED DESCRIPTION OF THE INVENTION

### Definitions

The terms “zwitterionic state”, “free zwitterion” and “free base” are used herein interchangeably and refer to the compound of formula (Ia) wherein the acidic carboxylic acid functional group is deprotonated (negatively charged), while one of the basic amino functional groups is protonated (positively charged).

The term “pharmaceutical composition” refers to a mixture of a crystalline and/or amorphous form described herein, with other chemical components, such as carriers, stabilizers, diluents, dispersing agents, suspending agents, thickening agents, excipients, etc. The pharmaceutical composition facilitates administration of the compound to a mammal.

“Detectable amount” refers to an amount that is measurable using standard analytic methods (e.g. ion chromatography, mass spectrometry, NMR, HPLC, gas chromatography, elemental analysis, IR spectroscopy, inductively coupled plasma atomic emission spectrometry, USP<231>Method II, etc) (ICH guidances, Q2A Text on Validation of Analytical Procedures (March 1995) and Q2B Validation of Analytical Procedures: Methodology (November 1996)).

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of an agent being administered, which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. The term “therapeutically effective amount” includes, for example, a prophylactically effective amount. The effective amount will be selected based on the particular patient and the disease level. It is understood that “an effect amount” or “a therapeutically effective amount” varies from subject to subject, due to variation in metabolism of drug, age, weight, general condition of the subject, the condition being treated, the severity of the condition being treated, and the judgment of the prescribing physician. In one embodiment, an appropriate “effective” amount in any individual case is determined using techniques, such as a dose escalation study. In some embodiments, the term “effective amount” or “therapeutically effective amount,” is used in reference to the crystalline and amorphous forms described herein being administered, which will relieve to some extent one or more of the symptoms of the disease or condition being treated.

The terms “kit” and “article of manufacture” are used as synonyms.

### Crystalline and Amorphous Forms

In a first aspect, the present invention provides a mono hydrochloric acid salt of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula I, “HCl salt”).

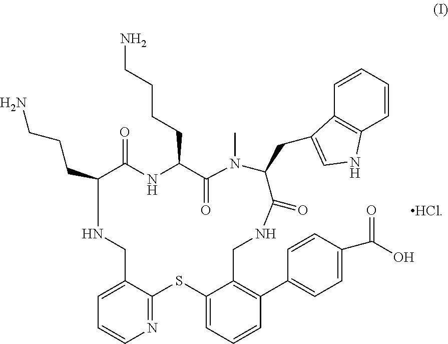

The HCl salt of the invention displays a dramatically increased solubility compared to the free base, especially in aqueous media, which is of utmost importance for intravenous administration (Example 14).

In one embodiment, the HCl salt of the invention is crystalline and has an X-ray powder diffraction (XRPD) pattern comprising peaks at 5.16, 7.94, 9.98, 10.46, 10.78, 11.66, 17.28, and 18.90 [° 2 Theta±0.2° 2 Theta, Cu Kα radiation (1.5406 Å)].

In one embodiment, the HCl salt of the invention is crystalline and:
(a) has an X-ray powder diffraction (XRPD) pattern comprising peaks at 5.16, 7.94, 9.98, 10.46, 10.78, 11.66, 15.52, 15.70, 17.28, and 18.90 [° 2 Theta±0.2° 2 Theta, Cu Kα radiation (1.5406 Å)]; and/or
(b) has an FT Raman spectrum comprising absorption bands at wavenumbers of about 103 cm⁻¹, 1390 cm⁻¹, 2246 cm⁻¹, and 2930 cm⁻¹±2 cm⁻¹; and/or
(c) has an ATR-FTIR spectrum comprising absorption bands at wavenumbers of about 752 cm⁻¹, 1386 cm⁻¹, 1534 cm⁻¹, and 1602 cm⁻¹+2 cm⁻¹.

In a preferred embodiment, the HCl salt of the invention is crystalline and:
(a) has an X-ray powder diffraction (XRPD) pattern comprising peaks at 5.16, 6.54, 7.94, 9.98, 10.46, 10.78, 11.54, 11.66, 11.98, 12.10, 13.02, 13.16, 14.38, 14.82, 15.04, 15.52, 15.70, 15.94, 16.74, 17.28, 17.74, 18.10, 18.32, 18.78, 18.90, 19.72, 20.02, 20.28, 20.80, 20.96, 21.16, 21.56, 21.62, 22.38, 22.66, 23.28, 23.50, 23.98, 24.72, 24.92, 25.20, 25.60, 25.98, 26.22, 26.98, 27.20, 27.42, 27.88, 28.10, 28.48, 28.66, 29.00, 29.70, and 30.00 [° 2 Theta±0.2° 2 Theta, Cu Kα radiation (1.5406 Å)]; and/or
(b) has an FT Raman spectrum comprising absorption bands at wavenumbers of about 103 cm⁻¹, 137 cm⁻¹, 225 cm⁻¹, 279 cm⁻¹, 330 cm⁻¹, 382 cm⁻¹, 416 cm⁻¹, 449 cm⁻¹, 479 cm⁻¹, 517 cm⁻¹, 547 cm⁻¹, 563 cm⁻¹, 596 cm⁻¹, 624 cm⁻¹, 639 cm⁻¹, 685 cm⁻¹, 758 cm⁻¹, 790 cm⁻¹, 807 cm⁻¹, 848 cm⁻¹, 878 cm⁻¹, 898 cm⁻¹, 955 cm⁻¹, 1011 cm⁻¹, 1059 cm⁻¹, 1074 cm⁻¹, 1140 cm⁻¹, 1183 cm⁻¹, 1203 cm⁻¹, 1247 cm⁻¹, 1288 cm⁻¹, 1316 cm⁻¹, 1334 cm⁻¹, 1358 cm⁻¹, 1391 cm⁻¹, 1425 cm⁻¹, 1435 cm⁻¹, 1451 cm⁻¹, 1489 cm⁻¹, 1557 cm⁻¹, 1580 cm⁻¹, 1605 cm⁻¹, 1666 cm⁻¹, 2246 cm⁻¹, 2859 cm⁻¹, 2930 cm⁻¹, 2985 cm⁻¹, 3063 cm⁻¹, and 3116 cm⁻¹±2 cm⁻¹; and/or
(c) has an ATR-FTIR spectrum comprising absorption bands at wavenumbers of about 659 cm⁻¹, 717 cm⁻¹, 731 cm⁻¹, 752 cm⁻¹, 778 cm⁻¹, 786 cm⁻¹, 803 cm⁻¹, 847 cm⁻¹, 874 cm⁻¹, 887 cm⁻¹, 906 cm⁻¹, 931 cm⁻¹, 950 cm⁻¹, 971 cm⁻¹, 1010 cm⁻¹, 1017 cm⁻¹, 1080 cm⁻¹, 1098 cm⁻¹, 1134 cm⁻¹, 1148 cm⁻¹, 1180 cm⁻¹, 1215 cm⁻¹, 1248 cm⁻¹, 1282 cm⁻¹, 1334 cm⁻¹, 1385 cm⁻¹, 1439 cm⁻¹, 1459 cm⁻¹, 1534 cm⁻¹, 1602 cm⁻¹, 1659 cm⁻¹, 1682 cm⁻¹, 2936 cm⁻¹, 3054 cm⁻¹, 3239 cm⁻¹, and 3434 cm⁻¹±2 cm⁻¹.

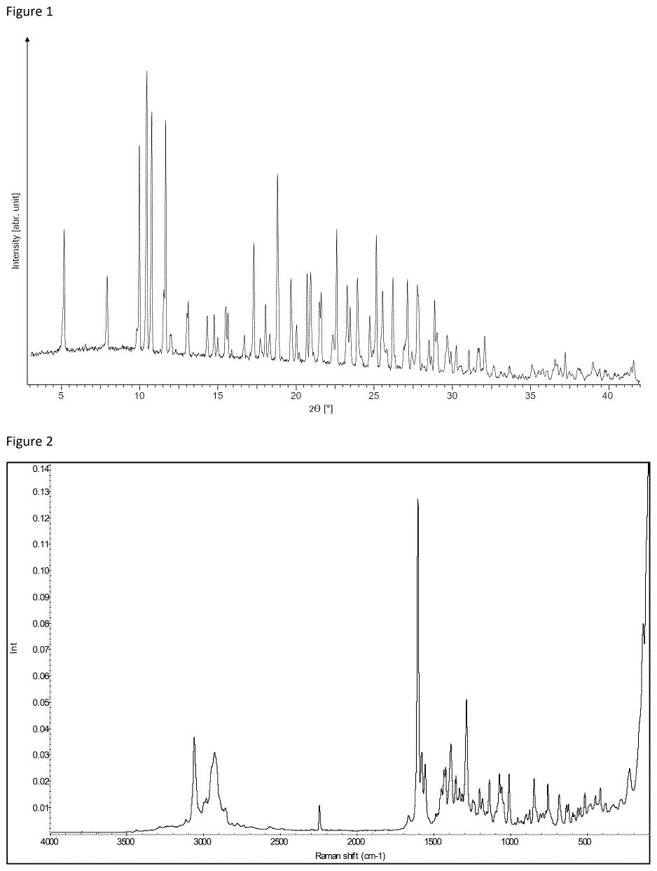

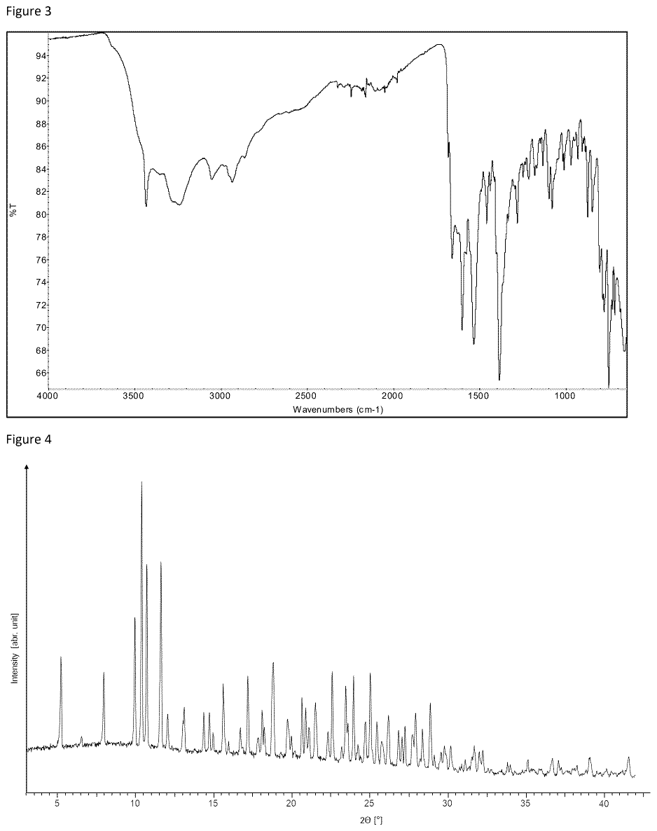

In a particularly preferred embodiment, the HCl salt of the invention is crystalline and:
(a) has an X-Ray powder diffraction (XRPD) pattern substantially the same as shown in FIG. 1; and/or
(b) has an FT Raman spectrum substantially the same as shown in FIG. 2; and/or
(c) has an ATR-FTIR spectrum substantially the same as shown in FIG. 3.

In one embodiment, the HCl salt of the invention is crystalline and:
(a) has an X-ray powder diffraction (XRPD) pattern comprising peaks at 5.20, 7.94, 9.92, 10.36, 10.68, 11.60, 15.58, 17.16, and 18.78 [° 2 Theta±0.2° 2 Theta, Cu Kα radiation (1.5406 Å)]; and/or
(b) has an FT Raman spectrum comprising absorption bands at wavenumbers of about 163 cm⁻¹, 1391 cm⁻¹, 2920 cm⁻¹, and 2948 cm⁻¹±2 cm⁻¹; and/or
(c) has an ATR-FTIR spectrum comprising absorption bands at wavenumbers of about 753 cm⁻¹, 1387 cm⁻¹, 1541 cm⁻¹, 1604 cm⁻¹±2 cm⁻¹.

In a preferred embodiment, the HCl salt of the invention is crystalline and:
(a) has an X-ray powder diffraction (XRPD) pattern comprising peaks at 5.20, 6.52, 7.94, 9.92, 10.36, 10.68, 11.60, 12.04, 13.02, 13.10, 14.34, 14.70, 14.92, 15.58, 15.94, 16.68, 17.16, 17.52, 17.82, 18.06, 18.22, 18.78, 19.70, 19.80, 19.96, 20.64, 20.88, 21.10, 21.50, 22.30, 22.58, 23.18, 23.44, 23.58, 23.94, 24.22, 24.70, 25.02, 25.42, 25.74, 26.18, 26.82, 27.04, 27.24, 27.72, 27.90, 28.34, 28.86, 29.10, 29.52, 29.74, 30.14, 31.08, and 31.52 [° 2 Theta±0.2° 2 Theta, Cu Kα radiation (1.5406 Å)]; and/or
(b) has an FT Raman spectrum comprising absorption bands at wavenumbers of about 107 cm⁻¹, 135 cm⁻¹, 163 cm⁻¹, 225 cm⁻¹, 336 cm⁻¹, 417 cm⁻¹, 450 cm⁻¹, 480 cm⁻¹, 518 cm⁻¹, 546 cm⁻¹, 562 cm⁻¹, 596 cm⁻¹, 621 cm⁻¹, 639 cm⁻¹, 686 cm⁻¹, 758 cm⁻¹, 774 cm⁻¹, 790 cm⁻¹, 813 cm⁻¹, 848 cm⁻¹, 877 cm⁻¹, 899 cm⁻¹, 955 cm⁻¹, 1011 cm⁻¹, 1046 cm⁻¹, 1059 cm⁻¹, 1075 cm⁻¹, 1140 cm⁻¹, 1188 cm⁻¹, 1200 cm⁻¹, 1234 cm⁻¹, 1249 cm⁻¹, 1289 cm⁻¹, 1317 cm⁻¹, 1335 cm⁻¹, 1358 cm⁻¹, 1391 cm⁻¹, 1426 cm⁻¹, 1435 cm⁻¹, 1452 cm⁻¹, 1489 cm⁻¹, 1558 cm⁻¹, 1578 cm⁻¹, 1606 cm⁻¹, 1660 cm⁻¹, 2920 cm⁻¹, 2948 cm⁻¹, 2986 cm⁻¹, and 3059 cm⁻¹±2 cm⁻¹; and/or
(c) has an ATR-FTIR spectrum comprising absorption bands at wavenumbers of about 673 cm⁻¹, 718 cm⁻¹, 739 cm⁻¹, 753 cm⁻¹, 773 cm⁻¹, 779 cm⁻¹, 803 cm⁻¹, 848 cm⁻¹, 875 cm⁻¹, 888 cm⁻¹, 898 cm⁻¹, 906 cm⁻¹, 932 cm⁻¹, 946 cm⁻¹, 971 cm⁻¹, 1010 cm⁻¹, 1017 cm⁻¹, 1037 cm⁻¹, 1067 cm⁻¹, 1082 cm⁻¹, 1095 cm⁻¹, 1134 cm⁻¹, 1149 cm⁻¹, 1169 cm⁻¹, 1181 cm⁻¹, 1223 cm⁻¹, 1248 cm⁻¹, 1286 cm⁻¹, 1333 cm⁻¹, 1387 cm⁻¹, 1406 cm⁻¹, 1424 cm⁻¹, 1440 cm⁻¹, 1459 cm⁻¹, 1467 cm⁻¹, 1541 cm⁻¹, 1585 cm⁻¹, 1604 cm⁻¹, 1658 cm⁻¹, 2937 cm⁻¹, 3054 cm⁻¹, 3230 cm⁻¹, 3349 cm⁻¹, and 3429 cm⁻¹±2 cm⁻¹.

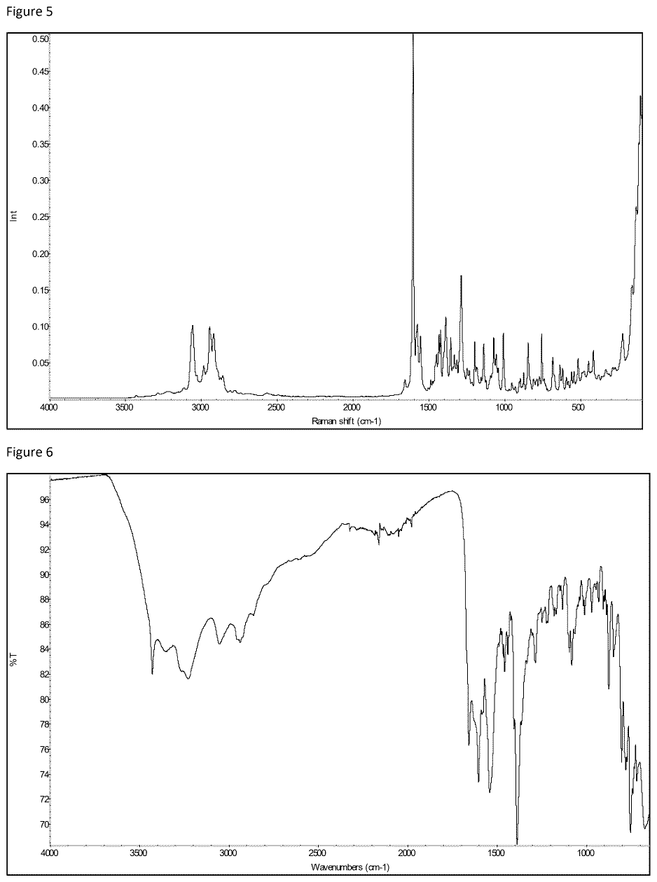

In a particularly preferred embodiment, the HCl salt of the invention is crystalline and:
(a) has an X-Ray powder diffraction (XRPD) pattern substantially the same as shown in FIG. 4; and/or
(b) has an FT Raman spectrum substantially the same as shown in FIG. 5; and/or
(c) has an ATR-FTIR spectrum substantially the same as shown in FIG. 6.

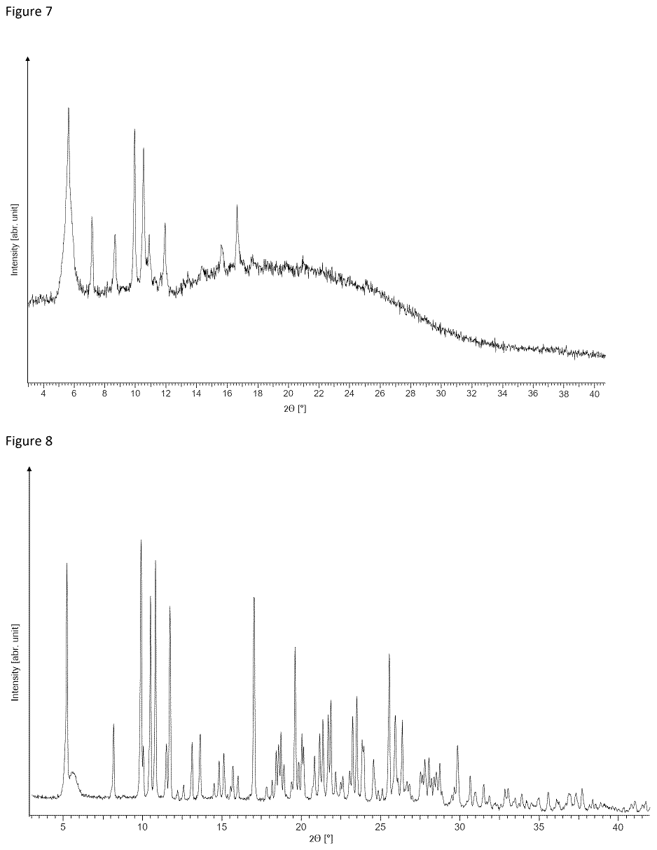

In one embodiment, the HCl salt of the invention is crystalline and has an X-ray powder diffraction (XRPD) pattern comprising peaks at 5.6, 7.14, 8.66, 9.92, 10.52, 10.88, 11.92, 15.6, 16.64, 17.7, and 20.94 [° 2 Theta±0.2° 2 Theta, Cu Kα radiation (1.5406 Å)]. In a preferred embodiment, the HCl salt of the invention is crystalline and has an X-ray powder diffraction (XRPD) pattern substantially the same as shown in FIG. 7.

In one embodiment, the HCl salt of the invention is crystalline and has an X-ray powder diffraction (XRPD) pattern comprising peaks at 5.22, 8.16, 9.88, 10.04, 10.48, 10.8, 11.48, 11.72, 12.2, 12.58, 13.1, 13.62, 14.5, 14.82, 15.12, 15.7, 16.02, 17.02, 17.82, 18.16, 18.42, 18.58, 18.72, 18.9, 19.4, 19.62, and 19.84 [° 2 Theta±0.2° 2 Theta, Cu Kα radiation (1.5406 Å)].

In a preferred embodiment, the HCl salt of the invention is crystalline and has an X-ray powder diffraction (XRPD) pattern substantially the same as shown in FIG. 8. In one embodiment, the HCl salt of the invention is amorphous and:
(a) has an FT Raman spectrum comprising absorption bands at wavenumbers of about 1296 cm⁻¹, 1608 cm⁻¹, 2928 cm⁻¹, and 3060 cm⁻¹±2 cm⁻¹±2 cm⁻¹; and/or
(b) has an ATR-FTIR spectrum comprising absorption bands at wavenumbers of about 742 cm⁻¹, 778 cm⁻¹, 1377 cm⁻¹, and 1531 cm⁻¹±2 cm⁻¹.

In a preferred embodiment, the HCl salt of the invention is amorphous and:
(a) has an FT Raman spectrum comprising absorption bands at wavenumbers of about 334 cm⁻¹, 415 cm⁻¹, 543 cm⁻¹, 641 cm⁻¹, 683 cm⁻¹, 759 cm⁻¹, 803 cm⁻¹, 842 cm⁻¹, 879 cm⁻¹, 912 cm⁻¹, 1011 cm⁻¹, 1061 cm⁻¹, 1077 cm⁻¹, 1137 cm⁻¹, 1203 cm⁻¹, 1296 cm⁻¹, 1359 cm⁻¹, 1381 cm⁻¹, 1437 cm⁻¹, 1453 cm⁻¹, 1557 cm⁻¹, 1579 cm⁻¹, 1609 cm⁻¹, 2928 cm⁻¹, and 3059 cm⁻¹±2 cm⁻¹; and/or
(b) has an ATR-FTIR spectrum comprising absorption bands at wavenumbers of about 680 cm⁻¹, 716 cm⁻¹, 742 cm⁻¹, 778 cm⁻¹, 841 cm⁻¹, 868 cm⁻¹, 1010 cm⁻¹, 1043 cm⁻¹, 1078 cm⁻¹, 1129 cm⁻¹, 1177 cm⁻¹, 1224 cm⁻¹, 1285 cm⁻¹, 1376 cm⁻¹, 1403 cm⁻¹, 1456 cm⁻¹, 1531 cm⁻¹, 1582 cm⁻¹, 1627 cm⁻¹, 2924 cm⁻¹, and 3243 cm⁻¹±2 cm⁻¹.

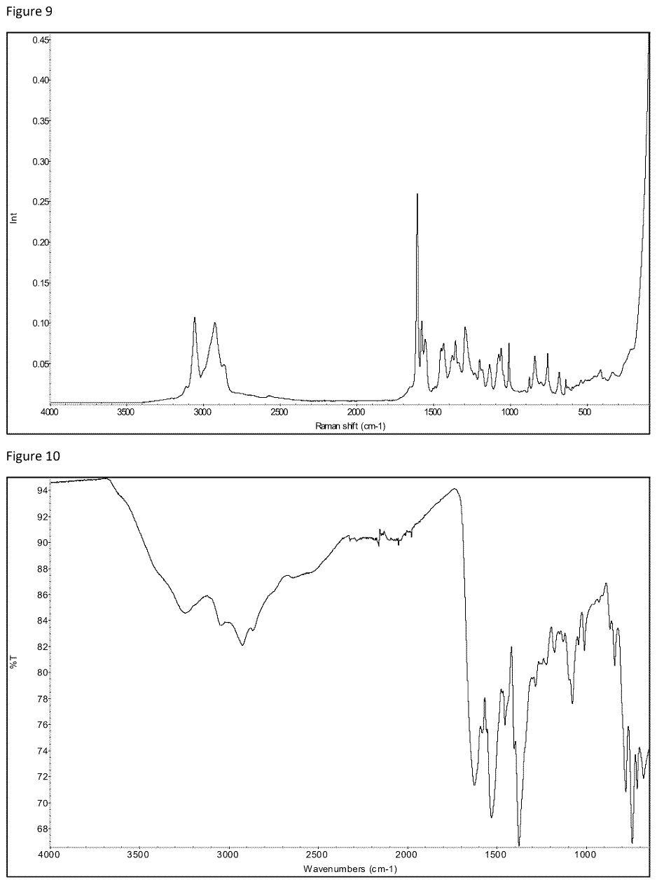

In a particularly preferred embodiment, the HCl salt of the invention is amorphous and:
(a) has an FT Raman spectrum substantially the same as shown in FIG. 9; and/or
(b) has an ATR-FTIR spectrum substantially the same as shown in FIG. 10.

In a further aspect, the present invention provides the free base, i.e., free zwitterion, of 4-[(11 S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula Ia).

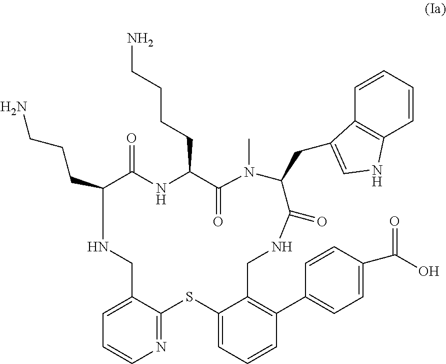 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

In one embodiment, the free base of the invention is crystalline.

In one embodiment, the free base of the invention is amorphous.

In one embodiment, the free base of the invention is crystalline and:
(a) has an X-ray powder diffraction (XRPD) pattern comprising peaks at 5.46, 8.08, 10.36, 11.56, 11.86, 16.56 and 17.44 [° 2 Theta±0.2° 2 Theta, Cu Kα radiation (1.5406 Å)]; and/or
(b) has an FT Raman spectrum comprising absorption bands at wavenumbers of about 838 cm⁻¹, 1079 cm⁻¹, 1373 cm⁻¹, and 1608 cm⁻¹±2 cm⁻¹; and/or
(c) has an ATR-FTIR spectrum comprising absorption bands at wavenumbers of about 746 cm⁻¹, 784 cm⁻¹, 1371 cm⁻¹, and 3437 cm⁻¹±2 cm⁻¹.

In a preferred embodiment, the free base of the invention is crystalline and:
(a) has an X-ray powder diffraction (XRPD) pattern comprising peaks at 5.46, 8.08, 9.90, 10.36, 11.56, 11.86, 12.46, 12.84, 13.36, 13.70, 13.86, 14.82, 14.98, 15.28, 16.56, 17.00, 17.44, 17.72, 18.18, 18.42, 18.64, 18.94, 19.26, 19.80, 20.12, 20.52, 20.78, 21.02, 21.14, 21.34, 21.48, 21.66, 21.80, 22.74, 22.84, 23.22, 23.42, 23.72, 24.02, 24.42, 24.58, 25.10, 25.52, 25.92, 26.18, 26.96, 27.60, 27.82, 27.94, 28.64, 29.16, and 29.92 [° 2 Theta±0.2° 2 Theta, Cu Kα radiation (1.5406 Å)]; and/or
(b) has an FT Raman spectrum comprising absorption bands at wavenumbers of about 233 cm⁻¹, 334 cm⁻¹, 415 cm⁻¹, 490 cm⁻¹, 540 cm⁻¹, 575 cm⁻¹, 596 cm⁻¹, 625 cm⁻¹, 642 cm⁻¹, 680 cm⁻¹, 699 cm⁻¹, 719 cm⁻¹, 757 cm⁻¹, 791 cm⁻¹, 810 cm⁻¹, 838 cm⁻¹, 879 cm⁻¹, 899 cm⁻¹, 1010 cm⁻¹, 1060 cm⁻¹, 1080 cm⁻¹, 1134 cm⁻¹, 1204 cm⁻¹, 1234 cm⁻¹, 1288 cm⁻¹, 1345 cm⁻¹, 1359 cm⁻¹, 1374 cm⁻¹, 1437 cm⁻¹, 1556 cm⁻¹, 1576 cm⁻¹, 1608 cm⁻¹, 2867 cm⁻¹, 2928 cm⁻¹, 3062 cm⁻¹, and 3114 cm⁻¹±2 cm⁻¹; and/or
(c) has an ATR-FTIR spectrum comprising absorption bands at wavenumbers of about 676 cm⁻¹, 716 cm⁻¹, 746 cm⁻¹, 774 cm⁻¹, 784 cm⁻¹, 838 cm⁻¹, 868 cm⁻¹, 904 cm⁻¹, 933 cm⁻¹, 1009 cm⁻¹, 1043 cm⁻¹, 1077 cm⁻¹, 1125 cm⁻¹, 1175 cm⁻¹, 1216 cm⁻¹, 1249 cm⁻¹, 1286 cm⁻¹, 1301 cm⁻¹, 1370 cm⁻¹, 1457 cm⁻¹, 1470 cm⁻¹, 1533 cm⁻¹, 1587 cm⁻¹, 1651 cm⁻¹, 2863 cm⁻¹, 2927 cm⁻¹, 3041 cm⁻¹, 3223 cm⁻¹, and 3437 cm^{÷1}±2 cm⁻¹.

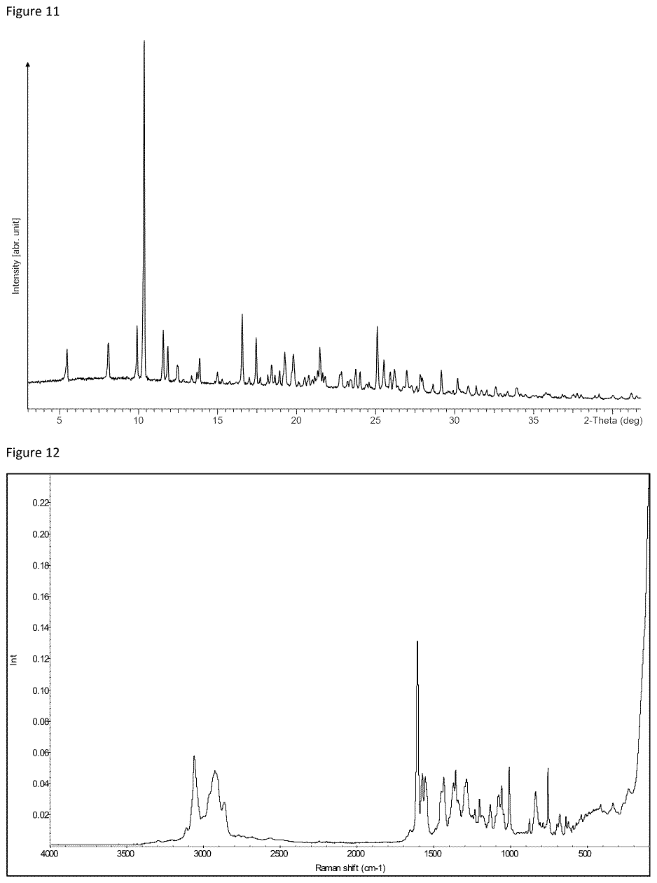

In a particularly preferred embodiment, the free base of the invention is crystalline and:
(a) has an X-Ray powder diffraction (XRPD) pattern substantially the same as shown in FIG. 11; and/or
(b) has an FT Raman spectrum substantially the same as shown in FIG. 12; and/or
(c) has an ATR-FTIR spectrum substantially the same as shown in FIG. 13.

It has surprisingly been found that a crystalline “form B” of the free base greatly facilitates the purification of the drug substance, allowing manufacture on an industrial scale under GMP conditions. Thus, crystalline “form B” described in Example 10 shows significantly reduced levels of palladium and sodium chloride contamination compared to the amorphous form described in Example 7. Thus, providing the API as a free base and crystallizing it in form B is a useful strategy for avoiding tedious nanofiltration to remove sodium chloride, as well as further inefficient purification steps to reduce palladium contamination. Form B of the free base is therefore highly useful in the manufacture of the API.

In one embodiment, the free base of the invention is crystalline and:
(a) has an X-ray powder diffraction (XRPD) pattern comprising peaks at 6.6, 9.0, 9.9, 10.1, 11.8, 12.0, 14.5, 15.4 [° 2 Theta±0.2° 2 Theta, Cu Kα radiation (1.5406 Å)]; and/or
(b) has an FT Raman spectrum comprising absorption bands at wavenumbers of about 757 cm⁻¹, 1290 cm⁻¹, 1433 cm⁻¹, and 1548 cm⁻¹±2 cm⁻¹; and/or
(c) has an ATR-FTIR spectrum comprising absorption bands at wavenumbers of about 745 cm⁻¹, 1367 cm⁻¹, 1533 cm⁻¹, and 1638 cm⁻¹±2 cm⁻¹.

In a preferred embodiment, the free base of the invention is crystalline and:
(a) has an X-ray powder diffraction (XRPD) pattern comprising peaks at 6.6, 9.0, 9.9, 10.1, 11.8, 12.0, 14.5, 14.8, 15.4, 15.7, 16.0, 16.4, 16.7, 17.0, 17.8, 18.0, 18.4, 18.7, 19.1, 19.6, 20.0, 20.4, 20.7, 20.9, 21.2, 21.5, 22.1, 23.3, 23.8, 24.2, 24.6, 24.9 and 25.7 [° 2 Theta±0.2° 2 Theta, Cu Kα radiation (1.5406 Å)]; and/or
(b) has an FT Raman spectrum comprising absorption bands at wavenumbers of about 233 cm⁻¹, 331 cm⁻¹, 352 cm⁻¹, 414 cm⁻¹, 462 cm⁻¹, 543 cm⁻¹, 574 cm⁻¹, 596 cm⁻¹, 614 cm⁻¹, 625 cm⁻¹, 641 cm⁻¹, 682 cm⁻¹, 696 cm⁻¹, 718 cm⁻¹, 757 cm⁻¹, 839 cm⁻¹, 878 cm⁻¹, 947 cm⁻¹, 1010 cm⁻¹, 1044 cm⁻¹, 1059 cm⁻¹, 1075 cm⁻¹, 1097 cm⁻¹, 1136 cm⁻¹, 1149 cm⁻¹, 1179 cm⁻¹, 1203 cm⁻¹, 1234 cm⁻¹, 1290 cm⁻¹, 1303 cm⁻¹, 1361 cm⁻¹, 1372 cm⁻¹, 1433 cm⁻¹, 1453 cm⁻¹, 1491 cm⁻¹, 1547 cm⁻¹, 1557 cm⁻¹, 1576 cm⁻¹, 1610 cm⁻¹, 1660 cm⁻¹, 2868 cm⁻¹, 2930 cm⁻¹, 3059 cm⁻¹, and 3113 cm⁻¹±2 cm⁻¹; and/or
(c) has an ATR-FTIR spectrum comprising absorption bands at wavenumbers of about 677 cm⁻¹, 716 cm⁻¹, 745 cm⁻¹, 771 cm⁻¹, 784 cm⁻¹, 801 cm⁻¹, 839 cm⁻¹, 868 cm⁻¹, 906 cm⁻¹, 927 cm⁻¹, 967 cm⁻¹, 1014 cm⁻¹, 1043 cm⁻¹, 1077 cm⁻¹, 1125 cm⁻¹, 1171 cm-1, 1213 cm⁻¹, 1243 cm⁻¹, 1283 cm⁻¹, 1301 cm⁻¹, 1367 cm⁻¹, 1399 cm⁻¹, 1431 cm⁻¹, 1456 cm⁻¹, 1471 cm⁻¹, 1533 cm⁻¹, 1590 cm⁻¹, 1638 cm⁻¹, 1658 cm⁻¹, 1694 cm⁻¹, 2862 cm⁻¹, 2925 cm⁻¹, 3029 cm⁻¹, and 3212 cm⁻¹±2 cm⁻¹.

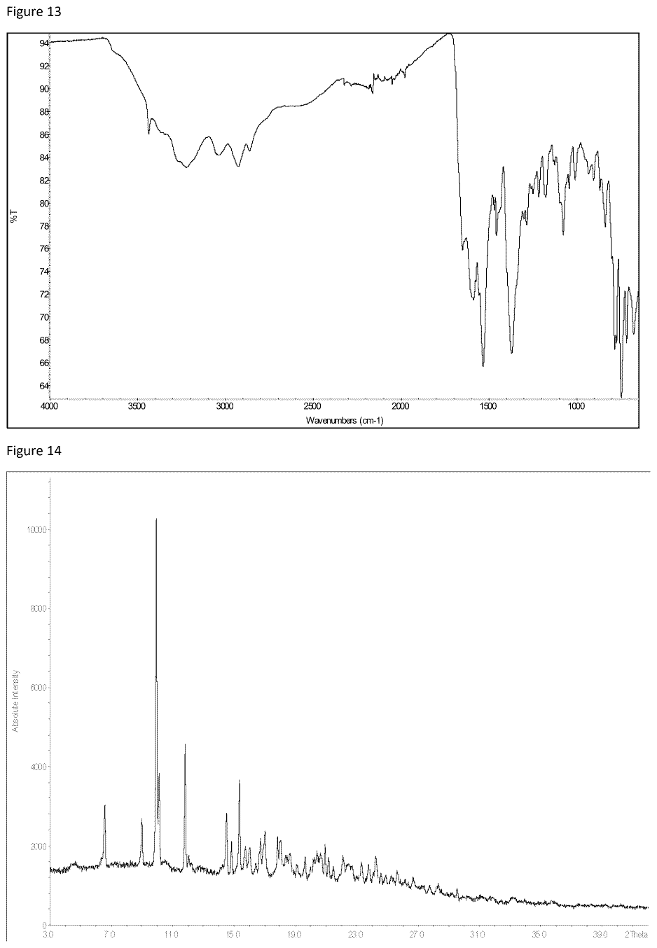

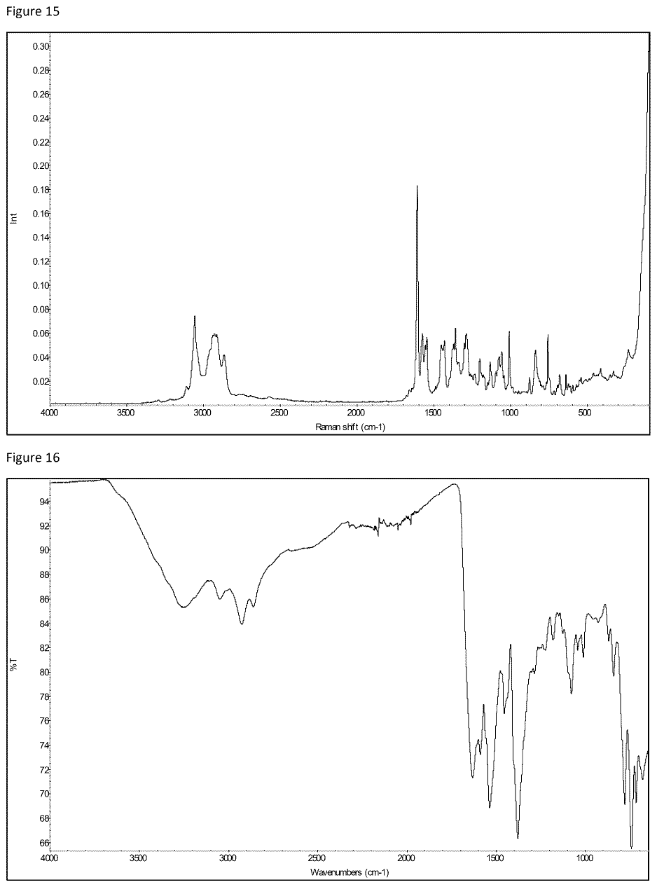

In a particularly preferred embodiment, the free base of the invention is crystalline and:
(a) has an X-Ray powder diffraction (XRPD) pattern substantially the same as shown in FIG. 14; and/or
(b) has an FT Raman spectrum substantially the same as shown in FIG. 15; and/or
(c) has an ATR-FTIR spectrum substantially the same as shown in FIG. 16.

In one embodiment, the free base of the invention is amorphous and:
(a) has an FT Raman spectrum comprising absorption bands at wavenumbers of about 1296 cm⁻¹, 1608 cm⁻¹, 2928 cm⁻¹, and 3060 cm⁻¹±2 cm⁻¹±2 cm⁻¹; and/or
(b) has an ATR-FTIR spectrum comprising absorption bands at wavenumbers of about 742 cm⁻¹, 778 cm⁻¹, 1379 cm⁻¹, and 1536 cm⁻¹±2 cm⁻¹.

In a preferred embodiment, the free base of the invention is amorphous and:
(a) has an FT Raman spectrum comprising absorption bands at wavenumbers of about 335 cm⁻¹, 415 cm⁻¹, 544 cm⁻¹, 641 cm⁻¹, 684 cm⁻¹, 759 cm⁻¹, 801 cm⁻¹, 845 cm⁻¹, 879 cm⁻¹, 910 cm⁻¹, 1011 cm⁻¹, 1061 cm⁻¹, 1077 cm⁻¹, 1137 cm⁻¹, 1203 cm⁻¹, 1296 cm⁻¹, 1359 cm⁻¹, 1386 cm⁻¹, 1438 cm⁻¹, 1556 cm⁻¹, 1579 cm⁻¹, 1608 cm⁻¹, 2862 cm⁻¹, 2927 cm⁻¹, and 3060 cm⁻¹±2 cm⁻¹; and/or
(b) has an ATR-FTIR spectrum comprising absorption bands at wavenumbers of about 679 cm⁻¹, 716 cm⁻¹, 742 cm⁻¹, 778 cm⁻¹, 842 cm⁻¹, 869 cm⁻¹, 928 cm⁻¹, 1010 cm⁻¹, 1043 cm⁻¹, 1078 cm⁻¹, 1127 cm⁻¹, 1182 cm⁻¹, 1224 cm⁻¹, 1285 cm⁻¹, 1379 cm⁻¹, 1455 cm⁻¹, 1537 cm⁻¹, 1589 cm⁻¹, 1632 cm⁻¹, 2863 cm⁻¹, 2926 cm⁻¹, 3047 cm⁻¹, and 3255 cm⁻¹±2 cm⁻¹.

In a particularly preferred embodiment, the free base of the invention is amorphous and:
(a) has an FT Raman spectrum substantially the same as shown in FIG. 9; and/or
(b) has an ATR-FTIR spectrum substantially the same as shown in FIG. 17.

In one embodiment, the free base of the invention is crystalline and has an X-ray powder diffraction (XRPD) pattern comprising peaks at 6.93, 7.28, 12.11, 14.52, 15.04, 15.73, 19.44, and 22.00 [° 2 Theta±0.2° 2 Theta, Cu Kα radiation (1.5406 Å)].

In a preferred embodiment, the free base of the invention is crystalline and has an X-ray powder diffraction (XRPD) pattern comprising peaks at 6.93, 7.28, 8.07, 10.08, 12.11, 13.53, 13.92, 14.52, 15.04, 15.73, 16.21, 16.96, 17.57, 18.13, 18.46, 19.31, 19.44, 19.87, 20.25, 20.51, 20.97, 22.00, 22.29, 22.49, 22.93, 23.12, 23.33, 23.60, 24.03, 24.40, 24.92, 25.04, 25.26, 25.62, 25.86, 26.15, 26.52, 27.26, 28.66, and 30.58 [° 2 Theta±0.2° 2 Theta, Cu Kα radiation (1.5406 Å)].

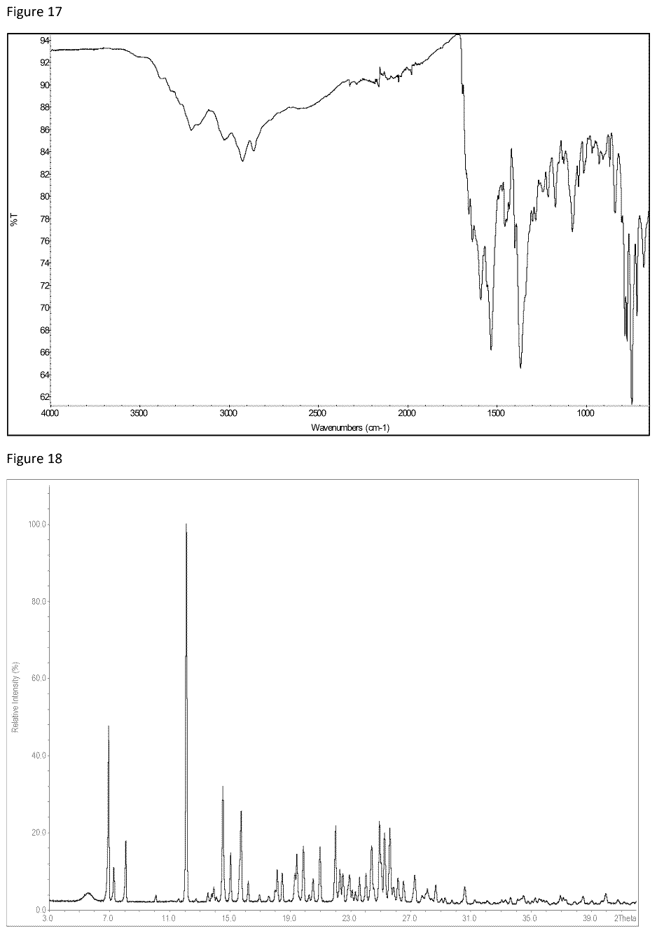

In a particularly preferred embodiment, the free base of the invention is crystalline and has an X-ray powder diffraction (XRPD) pattern substantially the same as shown in FIG. 18.

### Preparation of Crystalline Forms

In one aspect, the present invention provides processes for preparing the crystalline and amorphous forms described herein, wherein said processes are as outlined in the Examples. It is noted that solvents, temperatures and other reaction conditions presented in the Examples may vary.

In a further aspect, the present invention provides crystalline and amorphous forms described herein, when obtained by the processes described in the Examples.

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

In some embodiments, compositions comprising the crystalline and amorphous forms described herein include a residual amount of an organic solvent(s). In some embodiments, compositions comprising the crystalline and amorphous forms described herein include a detectable amount of an organic solvent(s). In some embodiments, compositions comprising the crystalline and amorphous forms described herein include a residual amount of a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from the group consisting of 1-butanol, 2-butanol, ethanol, 3-methyl-1-butanol, 2-methyl-1-propanol, 1-pentanol, 1-propanol, and 2-propanol. In some embodiments, the Class 3 solvent is ethanol or 1-propanol.

The methods and compositions described herein include the use the crystalline and amorphous forms described herein. In addition, the crystalline and amorphous forms described herein can exist in unsolvated as well as solvated forms with pharmaceutically acceptable solvents such as water, 1-propanol, ethanol, and the like.

### Pharmaceutical Compositions/Formulations

Pharmaceutical compositions are formulated in a conventional manner using one or more physiologically acceptable carriers comprising excipients and auxiliaries which facilitate processing of the active compounds into preparations which are used pharmaceutically. Suitable techniques, carriers, and excipients include those found within, for example, Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

In one aspect, the present invention relates to a pharmaceutical composition comprising any of the crystalline and amorphous forms described herein, or a mixture thereof, and at least one additional ingredient selected from pharmaceutically acceptable carriers, diluents and excipients.

In some embodiments, a crystalline or amorphous form described herein is formulated for intravenous administration to a mammal.

In one aspect, the present invention provides a solution for intravenous administration to a mammal, comprising:
(i) any of the crystalline and amorphous forms described herein, or a mixture thereof,
(ii) water for injection; and
(iii) sodium chloride.

In one embodiment, the concentration of the crystalline and amorphous forms described herein, or a mixture thereof, in the solution for intravenous administration according to the invention is 50 mg/mL.

Contemplated pharmaceutical compositions provide a therapeutically effective amount of the crystalline and amorphous forms described herein, enabling, for example, once-a-day, twice-a-day, three times a day, etc. administration. In one embodiment, pharmaceutical compositions provide an effective amount of the crystalline and amorphous forms described herein, enabling once-a-day dosing.

In one embodiment, the pharmaceutical compositions described herein are administered for therapeutic treatments. In therapeutic applications, the compositions are administered to a patient already suffering from a disease or condition, in an amount sufficient to cure or at least partially arrest at least one of the symptoms of the disease or condition. In certain embodiments, amounts effective for this use depend on the severity and course of the disease or condition, previous therapy, the patient's health status, weight, and response to the drugs, and/or the judgment of the treating physician.

### Using the Crystalline and Amorphous Forms of the Invention

The compounds described herein possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

In one aspect, the present invention provides the compounds described herein for use as a medicament.

In one embodiment, said medicament is an antibiotic.

In one aspect, the present invention provides a method of treating bacterial infections and resulting diseases in a mammal, said method comprising administering a therapeutically effective amount of a compound described herein to said mammal.

In one aspect, the present invention provides a compound described herein for use in the treatment of bacterial infections and resulting diseases in a mammal.

In one aspect, the present invention provides the use of a compound described herein for the treatment of bacterial infections and resulting diseases in a mammal.

In one aspect, the present invention provides the use of a compound described herein in the manufacture of a medicament for the treatment of bacterial infections and resulting diseases in a mammal.

In one embodiment, said resulting diseases are selected from bacteremia, pneumonia, meningitis, urinary tract infection, and wound infection.

In one embodiment, said bacterial infections are selected from infections with Gram-negative bacteria.

In one embodiment, said bacterial infections are selected from infections with an ‘ESKAPE’ pathogen (Enterococcus faecium, Staphylococcus aureus, Klebsiella pneumoniae, Acinetobacter baumannii, Pseudomonas aeruginosa and Enterobacter species & E. coli), or a combination thereof.

In one embodiment, said bacterial infections are nosocomial infections.

In one embodiment, said bacterial infections are selected from infections with Muti-Drug Resistant (MDR) bacteria, in particular by MDR A. baumannii.

In one embodiment, said bacterial infections are selected from infections with Carbapenem resistant bacteria, in particular Carbapanem resistant A. baumannii.

In one embodiment, said bacterial infections are selected from infections with Acinetobacter species, most particularly with Acinetobacter baumannii.

### Combination Treatments

The crystalline and amorphous forms described herein may be employed alone or in combination with other agents for treatment. For example, the second agent of the pharmaceutical combination formulation or dosing regimen may have complementary activities to the crystalline and amorphous forms described herein such that they do not adversely affect each other. The compounds may be administered together in a unitary pharmaceutical composition or separately. In one embodiment the crystalline and amorphous forms described herein can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination therof.

The term “co-administering” refers to either simultaneous administration, or any manner of separate sequential administration, of the crystalline and amorphous forms described herein and a further active pharmaceutical ingredient or ingredients, in particular antibiotic agents. If the administration is not simultaneous, the compounds are administered in a close time proximity to each other. Furthermore, it does not matter if the compounds are administered in the same dosage form, e.g. one compound may be administered intravenously and another compound may be administered orally.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), and macrolides (erythromycin).

In one aspect, the present invention provides a pharmaceutical composition described herein, further comprising an additional therapeutic agent.

In one aspect, the present invention provides a pharmaceutical combination comprising the crystalline and amorphous forms described herein and an additional therapeutic agent.

In one embodiment, said additional therapeutic agent is an antibiotic agent.

In one embodiment, said additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, said additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), and macrolides (erythromycin).

### EXAMPLES

The following examples are provided for illustration of the invention. They should not be considered as limiting the scope of the invention, but merely as being representative thereof.

### Abbreviations

The following abbreviations are used in the present patent specification:
ATR Attenuated total reflectance
FT Fourier transform
IR Infrared
XRPD X-Ray Powder Diffraction

### Example 1—Preparation of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (“free base”)

4-[(11S,14S,17S)-14-(4-aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid tetrakis(trifluoroacetate) salt (described in WO2019206853, 5.19 g, 4.2 mmol) was dissolved in water (60 mL) and acetonitrile (120 mL). Aqueous NaOH 32% (1.88 g) was added. To the solution was added NaOH aqueous 1 M until pH=9.8 was reached (ca 2 g). After a few hours, a suspension forms that is stirred for additional 1.5 hour at room temperature. The precipitate is filtered off, washed with water and dried under reduced pressure at 40° C. to afford 4-[(11S,14S,17S)-14-(4-aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (1.90 g, 59%) as a white powder.

MS: 791.37 [M+H⁺]

### Example 2—Preparation of amorphous 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid, mono hydrochloric acid salt (“HCl Salt”)

4-[(11S,14S,17S)-14-(4-aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (48.7 g, 61.6 mmol) was suspended in water (500 mL) and the pH was brought to 7.0 by addition of a mixture of aqueous HCl 25% (9.04 g) and water (51.2 mL). The solution was filtered through a 3M™ ZETA PLUS™ filter then through a 0.2 μm Sartopore® 2XLG® filter. The clear solution obtained was spray-dried and dried under vacuum to afford 4-[(11S,14S,17S)-14-(4-aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid mono hydrochloride (38.8 g, 76%) as a white powder (amorphous form).

MS: 791.37 [M+H⁺]

### Example 3—Preparation of Crystalline Form A1 of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid, mono hydrochloric acid salt

200 mg of amorphous HCl salt (Example 2) were dissolved in 0.5 mL water at 23° C. while stirring to form a high viscous solution. Once no solid particles were visible anymore, acetonitrile was added dropwise to the solution. White clouds were observed in the solution that disappeared quickly (tiny oil droplets form these clouds). More acetonitrile was added until the clouds did not disappear anymore to adjust a very slight supersaturation. A spatula tip seeds of the crystalline free base (Form A1) was added to the solution. The seeds did not dissolve as observed by eye. The mixture was stirred at 200 rpm and 22.5° C. for 12 hours. The solid material was separated from the liquid and identified as crystalline material by optical microscopy. Subsequently the material was filtered and dried under reduced pressure (20 mbar) and room temperature (22.5° C.) for 14 hours. The white powder was characterized by XRPD as crystalline Form A1.

On a larger scale, 50.46 g of amorphous HCl salt (Example 2) were dissolved in 85 mL water at 23° C. while stirring. Once no solid particles were visible anymore, 150 mL acetonitrile were added slowly. After approximately 50 mL, the HCl salt started oiling out. The emulsion was stirred for approximately 30 minutes until spontaneous crystallization of the oily phase was observed. Additional 50 mL acetonitrile were added to maintain a stirrable suspension. The suspension was stirred for 24 hours. After filtration, the white powder was dried at reduced pressure (20 mbar) and room temperature (23° C.) for 14 hours. The resulting solid was identified as pure Form A1 by XRPD.

### Example 4—Preparation of Crystalline Form A2 of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid, mono hydrochloric acid salt

100 mg of amorphous HCl salt (Example 2) were dissolved in 0.25 mL water at 23° C. while stirring to form a high viscous solution. Once no solid particles were visible anymore, a spatula tip of crystalline Form A1 seeds (Example 3) was added. The seeds did not dissolve as observed by eye. The mixture was stirred at 200 rpm and 23° C. for 12 hours. Subsequently a white suspension was observed. The material was filtered and dried under reduced pressure (20 mbar) and room temperature (23° C.) for 14 hours. The white powder was characterized by XRPD, Raman and IR as crystalline Form A2, being isostructural to Form A1.

On a larger scale, 2.02 g of amorphous HCl salt (Example 2) were dissolved in 3.00 mL water at 23° C. and stirred for 14 hours. The solution with extreme high viscosity was heated to 35° C. Seeds of form A2 were added, which rapidly dissolved. The solution was cooled to 2° C. overnight. The viscosity was too high to stir the clear, honey like solution. Temperature cycles between 10° C. and 35° C. were initiated and the sample was left while stirring at 200 rpm for 7 days. A white suspension was observed. The mixture was cooled to 5° C. and stirred at this temperature overnight. The solid was filtered and dried at ambient air (31% rH, 22° C.) for 6 hours. The white powder was characterized by XRPD as pure Form A2.

### Example 5—Preparation of Crystalline Form “Pattern 4” of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid, mono hydrochloric acid salt

Pattern 4 was observed by drying Form A2 (Example 4) at 50° C. and 5 mbar for 24 hours.

### Example 6—Preparation of Crystalline Form “Pattern 5” of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid, mono hydrochloric acid salt

Pattern 5 was observed by milling Form A1 (Example 3) in acetonitrile with glass beads in a vial on a vortex mixer.

### Example 7—Preparation of amorphous 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid

In a 1000 ml round bottom flask, 11.0 g amorphous HCl salt (Example 2), 323.5 ml acetone and 161.8 ml water were added at room temperature. The mixture was stirred. NaOH 0.1M was added dropwise to the orange mixture at room temperature until a pH of 9.80 was reached. 145 ml of sodium hydroxide 0.1M were consumed. The solvent was evaporated on the rotary evaporator as far as possible. The remainder was deep frozen with dry ice and then freeze dried to afford the title compound (Pd contamination: 3261 ppm, Cl— contamination (from NaCl): 4.2% wt/wt).

### Example 8—Preparation of Crystalline Form A1 of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid

44.21 g of amorphous HCl salt (Example 2) is suspended at room temperature in 260.2 mL of water and 71.88 mL of acetonitrile. To the white suspension were added 27.61 mL of HCl (0.214 mol, 4 eq). The white suspension changed to a light yellow turbid solution. Upon heating to 40° C., the solution turned clear. At room temperature, a mixture consisting of 20.15 mL water and 20.15 mL NaOH (0.218 mol, 4.07 eq) was added and the pH was adjusted to about neutral pH (pH 7.19). The solution was optionally seeded with free base (form A family, 10.4 mg). Afterwards, the pH of the solution was adjusted to pH 9.8 by adding 54.9 g 1 N NaOH. The colorless solution became increasingly cloudy from pH 9.1 onwards. The resulting suspension was stirred at room temperature for one hour and then filtered. The crystals were dried at 65° C./2 mbar overnight and at 25° C. in a vacuum drying oven to afford 41.82 g of pure form A1. It was found that the free base can also exist as isostructural mixed solvate/hydrate crystalline systems containing various organic solvents and water in the crystal lattice, which are not described here. For example, such isostructural crystalline forms can be obtained from solvent mixtures consisting of water and ethanol, or water in propanol.

### Example 9—Preparation of Crystalline Form B of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid

19.775 g of amorphous HCl salt (Example 2) were added to a mixture of 100 mL water and 15.56 mL 1-PrOH and dissolved under stirring at 35° C. The pH of the solution was set to 9.2 by addition of 50 mL of a 8 wt. % aqueous NaOH solution and 135 mg ABX free base seeds were added as a suspension in 1 g water and 1 g 1-PrOH. The resulting suspension was aged for 60 min. Thereafter, the pH was increased to 9.8 by addition over 1 h of 75 mL of a 1 wt. % aqueous NaOH solution. The suspension was aged for 15 min and the temperature decreased to 20° C. within 1 h. After 15 min additional aging time, the suspension was filtered, the solid washed with water, and a sample characterized by XRPD as pure crystalline form A3. The remaining material was dried at 80° C. and 20 mbar for 16 h. The resulting white powder was characterized by XRPD as pure crystalline Form B.

### Example 10—Alternative Preparation of Crystalline Form B of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid

1.7 g of the amorphous free base (Example 7) was weighed into a 40 mL scale up glass and 20 mL of solvent (15% water in propanol v/v) was added with stirring (350 rpm). A sticky red film formed at the bottom. Subsequently, the solution was heated to 80° C. whereby the reddish substance did not dissolve. The hot solution was filtered through a 0.2 μm Satorius Nutsche filter. The filtrate became turbid and was heated to 80° C. again. The yellow solution was cooled from 80° C. to 10° C. within 8 h without stirring. A yellow oil remained which was seeded at 25° C. under stirring (350 rpm) with a spatula tip of Form A. The white suspension was filtered through a Satorius Nutsche 0.2 um filter. The filter cake was washed with 3×10 mL of fresh solvent and then and dried overnight at 50° C./5 mbar. (off-white, crystalline solid, Pd contamination: 2048 ppm, Cl⁻ contamination (from NaCl): 0.1% wt/wt).

### Example 11—Preparation of Crystalline Form “Pattern 10.1” of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid

1 g of the Form B free base was weighed into a 7.5 mL scale up glass and 5 mL of solvent (5% water in ethanol v/v) was added with stirring (100 rpm) at 20° C. After 2 days, the white suspension was centrifuged at 5000 rpm. The resulting white wet powder was characterized by XRPD as pure crystalline Pattern 10.1.

### Example 12—ATR FTIR

### Experimental Methodology

The ATR-FTIR spectra were recorded without any sample preparation using a ThermoNicolet iS5 FTIR spectrometer with ATR accessory. The spectral range is between 4000 cm⁻¹ and 650 cm⁻¹, resolution 2 cm⁻¹ and 50 co-added scans were collected (except for the spectrum of the Form A1 from the HCl salt, where 32 co-added scans were collected). Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

### Results

The crystalline forms A1, A2 and the amorphous form of the mono hydrochloric acid salt of Formula (I), as well as crystalline forms A1, B and the amorphous form of the free base of formula (Ia) were characterised by ATR FTIR as described above. The unique ATR FTIR peaks are presented in Tables 1-6. Characteristic ATR FTIR spectra are shown in FIGS. 3, 6, 10, 13, 16 and 17.

TABLE 1
Unique ATR FTIR peaks for Amorphous Free
Base (cm⁻¹ ± 2 cm⁻¹)

679
716
742
778
842
869
928
1010
1043
1078
1127
1182
1224
1285
1379
1455
1537
1589
1632
2863
2926
3047
3255

TABLE 2
Unique ATR FTIR peaks for Free Base Crystalline
Form A1 (cm⁻¹ ± 2 cm⁻¹)

676
716
746
774
784
838
868
904
933
1009
1043
1077
1125
1175
1216
1249
1286
1301
1370
1457
1470
1533
1587
1651
2863
2927
3041
3223
3437

TABLE 3
Unique ATR FTIR peaks for Free Base Crystalline
Form B (cm⁻¹ ± 2 cm⁻¹)

677
716
745
771
784
801
839
868
906
927
967
1014
1043
1077
1125
1171
1213
1243
1283
1301
1367
1399
1431
1456
1471
1533
1590
1638
1658
1694
2862
2925
3029
3212

TABLE 4
Unique ATR FTIR peaks for Amorphous HCl Salt (cm⁻¹ ± 2 cm⁻¹)

680
716
742
778
841
868
1010
1043
1078
1129
1177
1224
1285
1376
1403
1456
1531
1582
1627
2924
3243

TABLE 5
Unique ATR FTIR peaks for HCl Salt Crystalline
Form A1 (cm⁻¹ ± 2 cm⁻¹)

659
717
731
752
778
786
803
847
874
887
906
931
950
971
1010
1017
1080
1098
1134
1148
1180
1215
1248
1282
1334
1385
1439
1459
1534
1602
1659
1682
2936
3054
3239
3434

TABLE 6
Unique ATR FTIR peaks for HCl Salt Crystalline
Form A2 (cm⁻¹ ± 2 cm⁻¹)

673
718
739
753
773
779
803
848
875
888
898
906
932
946
971
1010
1017
1037
1067
1082
1095
1134
1149
1169
1181
1223
1248
1286
1333
1387
1406
1424
1440
1459
1467
1541
1585
1604
1658
2937
3054
3230
3349
3429

### Example 13—Raman Spectroscopy

### Experimental Methodology

The FT-Raman spectra were recorded without any sample preparation using a Bruker MultiRam FT-Raman spectrometer equipped with a liquid nitrogen cooled Germanium detector and 1064 nm NdYAG laser. The spectral range is between 4000 cm⁻¹ and 100 cm⁻¹, resolution 2 cm⁻¹ (except for the spectrum of the Form A1 from the HCl salt: 4 cm⁻¹) and 2048 co-added scans (except for the spectrum of the Form B from the free base: 1024 scans) were collected. The laser power was set to 300 mW (except for the spectrum of the Form A1 from the HCl salt: 100 mW) and Blackman-Harris 4-Term apodization was applied.

### Results

The crystalline forms A1, A2 and the amorphous form of the mono hydrochloric acid salt of Formula (I), as well as crystalline forms A1, B and the amorphous form of the free base of formula (Ia) were characterised by FT Raman spectroscopy as described above. The unique FT-Raman peaks are presented in Tables 7-11. Characteristic FT Raman spectra are shown in FIGS. 2, 5, 9, 12, and 15. It is noted that the amorphous forms of the free base and the HCl salt are not distinguishable by Raman spectroscopy.

TABLE 7
Unique FT Raman peaks for Amorphous Free Base and Amorphous
HCl Salt (cm⁻¹ ± 2 cm⁻¹)

335
415
544
641
684
759
801
845
879
910
1011
1061
1077
1137
1203
1296
1359
1386
1438
1556
1579
1608
2862
2927
3060

TABLE 8
Unique FT Raman peaks for Free Base Crystalline
Form A1 (cm⁻¹ ± 2 cm⁻¹)

233
334
415
490
540
575
596
625
642
680
699
719
757
791
810
838
879
899
1010
1060
1080
1134
1204
1234
1288
1345
1359
1374
1437
1556
1576
1608
2867
2928
3062
3114

TABLE 9
Unique FT Raman peaks for Free Base Crystalline
Form B (cm⁻¹ ± 2 cm⁻¹)

233
331
352
414
462
543
574
596
614
625
641
682
696
718
757
839
878
947
1010
1044
1059
1075
1097
1136
1149
1179
1203
1234
1290
1303
1361
1372
1433
1453
1491
1547
1557
1576
1610
1660
2868
2930
3059
3113

TABLE 10
Unique FT Raman peaks for HCl Salt Crystalline
Form A1 (cm⁻¹ ± 2 cm⁻¹)

103
137
225
279
330
382
416
449
479
517
547
563
596
624
639
685
758
790
807
848
878
898
955
1011
1059
1074
1140
1183
1203
1247
1288
1316
1334
1358
1391
1425
1435
1451
1489
1557
1580
1605
1666
2246
2859
2930
2985
3063
3116

TABLE 11
Unique FT Raman peaks for HCl Salt Crystalline
Form A2 (cm⁻¹ ± 2 cm⁻¹)

107
135
163
225
336
417
450
480
518
546
562
596
621
639
686
758
774
790
813
848
877
899
955
1011
1046
1059
1075
1140
1188
1200
1234
1249
1289
1317
1335
1358
1391
1426
1435
1452
1489
1558
1578
1606
1660
2920
2948
2986
3059

### Example 14—XRPD

### Experimental Methodology

X-ray diffraction patterns were recorded at ambient conditions in transmission geometry with a STOE STADI P diffractometer (Cu Kα radiation (1.5406 Å), primary Ge-monochromator, Mythen 1K silicon strip detector, angular range 3° to 42° 2Theta, 20 seconds measurement time per step). The samples were prepared and analyzed without further processing (e.g. grinding or sieving) of the substance.

Measurement and evaluation of the X-ray diffraction data was done using WinXPOW software (STOE & Cie GmbH, Darmstadt, Germany).

### Results

The crystalline forms A1, A2, patterns 4 and 5 of the mono hydrochloric acid salt of Formula (I), as well as crystalline forms A1 and B of the free base of formula (Ia) were characterised by XRPD as described above. The unique XRPD peaks of the crystalline forms are presented in Tables 12-17. Characteristic XRPD diffractograms of the crystalline forms are shown in FIGS. 1, 4, 7, 8, 11, and 14.

TABLE 12
Free Base Crystalline Form A1
Position [°2Θ]

5.46
8.08
9.90
10.36
11.56
11.86
12.46
12.84
13.36
13.70
13.86
14.82
14.98
15.28
16.56
17.00
17.44
17.72
18.18
18.42
18.64
18.94
19.26
19.80
20.12
20.52
20.78
21.02
21.14
21.34
21.48
21.66
21.80
22.74
22.84
23.22
23.42
23.72
24.02
24.42
24.58
25.10
25.52
25.92
26.18
26.96
27.60
27.82
27.94
28.64
29.16
29.92

TABLE 13
Free Base Crystalline Form B
Position [°2Θ]

6.56
8.98
9.94
10.1
11.82
12.04
14.49
14.84
15.35
15.72
16.01
16.4
16.72
16.99
17.83
18.03
18.35
18.65
19.1
19.62
20
20.4
20.66
20.93
21.17
21.45
22.08
23.29
23.78
24.24
24.56
24.89
25.65

TABLE 14
HCl Salt Crystalline Form A1
Position [°2Θ]

5.16
6.54
7.94
9.98
10.46
10.78
11.54
11.66
11.98
12.10
13.02
13.16
14.38
14.82
15.04
15.52
15.70
15.94
16.74
17.28
17.74
18.10
18.32
18.78
18.90
19.72
20.02
20.28
20.80
20.96
21.16
21.56
21.62
22.38
22.66
23.28
23.50
23.98
24.72
24.92
25.20
25.60
25.98
26.22
26.98
27.20
27.42
27.88
28.10
28.48
28.66
29.00
29.70
30.00

TABLE 15
HCl Salt Crystalline Form A2
Position [°2Θ]

5.20
6.52
7.94
9.92
10.36
10.68
11.60
12.04
13.02
13.10
14.34
14.70
14.92
15.58
15.94
16.68
17.16
17.52
17.82
18.06
18.22
18.78
19.70
19.80
19.96
20.64
20.88
21.10
21.50
22.30
22.58
23.18
23.44
23.58
23.94
24.22
24.70
25.02
25.42
25.74
26.18
26.82
27.04
27.24
27.72
27.90
28.34
28.86
29.10
29.52
29.74
30.14
31.08
31.52

TABLE 16
HCI Salt Crystalline “Pattern 4”
Position [°2Θ]

5.6
7.14
8.66
9.92
10.52
10.88
11.92
15.6
16.64
17.7
20.94

TABLE 17
HCl Salt Crystalline “Pattern 5”
Position [°2Θ]

5.22
8.16
9.88
10.04
10.48
10.8
11.48
11.72
12.2
12.58
13.1
13.62
14.5
14.82
15.12
15.7
16.02
17.02
17.82
18.16
18.42
18.58
18.72
18.9
19.4
19.62
19.84

TABLE 18
Free Base “Pattern 10.1”
Position [°2Θ]

| | | | | | |
|---|---|---|---|---|---|
| 6.93 | 15.04 | 19.44 | 22.93 | 25.26 | 6.93 |
| 7.28 | 15.73 | 19.87 | 23.12 | 25.62 | 7.28 |
| 8.07 | 16.21 | 20.25 | 23.33 | 25.86 | 8.07 |
| 10.08 | 16.96 | 20.51 | 23.6 | 26.15 | 10.08 |
| 12.11 | 17.57 | 20.97 | 24.03 | 26.52 | 12.11 |
| 13.53 | 18.13 | 22 | 24.4 | 27.26 | 13.53 |
| 13.92 | 18.46 | 22.29 | 24.92 | 28.66 | 13.92 |
| 14.52 | 19.31 | 22.49 | 25.04 | 30.58 | 14.52 |

### Example 15—Solubility Assessment

### Experimental Methodology

Approximately 1 g of substance was equilibrated in 8.5 mL of water at 20° C. for 2 days. 1 mL of suspension was centrifuged. The filtrate was analyzed by uPLC.

### Results

| | | |
|---|---|---|
| Form A family free zwitterion | <1 | mg/mL |
| Form A family HCl salt | 123.5 | mg/mL |
| Amorphous HCl salt | 177.6 | mg/mL |

1. A mono hydrochloric acid salt of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula I)

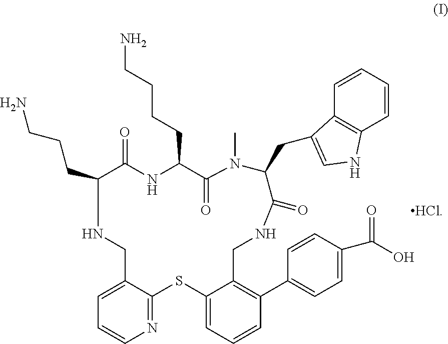

2. A zwitterion of 4-[(11S,14S,17S)-14-(4-Aminobutyl)-11-(3-aminopropyl)-17-(1H-indol-3-ylmethyl)-16-methyl-12,15,18-trioxo-2-thia-4,10,13,16,19-pentazatricyclo[19.4.0.03,8]pentacosa-1(25),3(8),4,6,21,23-hexaen-22-yl]benzoic acid (Formula Ia).

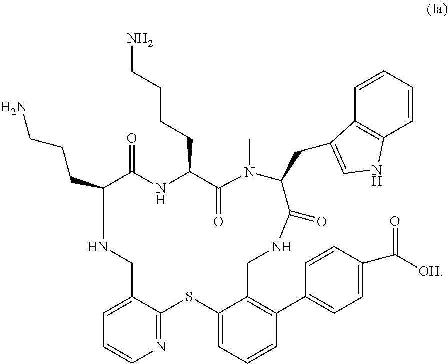 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

3. A zwitterion according to claim 2, wherein said zwitterion is in a crystalline form.

4. The crystalline form of claim 3, which:
(a) has an X-ray powder diffraction (XRPD) pattern comprising peaks at 6.6, 9.0, 9.9, 10.1, 11.8, 12.0, 14.5, 15.4 [° 2 Theta±0.2° 2 Theta, Cu Kα radiation (1.5406 Å)]; and/or
(b) has an FT Raman spectrum comprising absorption bands at wavenumbers of about 757 cm⁻¹, 1290 cm⁻¹, 1433 cm⁻¹, and 1548 cm⁻¹±2 cm⁻¹; and/or
(c) has an ATR-FTIR spectrum comprising absorption bands at wavenumbers of about 745 cm⁻¹, 1367 cm⁻¹, 1533 cm⁻¹, and 1638 cm⁻¹±2 cm⁻¹.

5. The crystalline form of claim 4, which:
(a) has an X-ray powder diffraction (XRPD) pattern comprising peaks at 6.6, 9.0, 9.9, 10.1, 11.8, 12.0, 14.5, 14.8, 15.4, 15.7, 16.0, 16.4, 16.7, 17.0, 17.8, 18.0, 18.4, 18.7, 19.1, 19.6, 20.0, 20.4, 20.7, 20.9, 21.2, 21.5, 22.1, 23.3, 23.8, 24.2, 24.6, 24.9 and 25.7 [° 2 Theta±0.2° 2 Theta, Cu Kα radiation (1.5406 Å)]; and/or
(b) has an FT Raman spectrum comprising absorption bands at wavenumbers of about 233 cm⁻¹, 331 cm⁻¹, 352 cm⁻¹, 414 cm⁻¹, 462 cm⁻¹, 543 cm⁻¹, 574 cm⁻¹, 596 cm⁻¹, 614 cm⁻¹, 625 cm⁻¹, 641 cm⁻¹, 682 cm⁻¹, 696 cm⁻¹, 718 cm⁻¹, 757 cm⁻¹, 839 cm⁻¹, 878 cm⁻¹, 947 cm⁻¹, 1010 cm⁻¹, 1044 cm⁻¹, 1059 cm⁻¹, 1075 cm⁻¹, 1097 cm⁻¹, 1136 cm⁻¹, 1149 cm⁻¹, 1179 cm⁻¹, 1203 cm⁻¹, 1234 cm⁻¹, 1290 cm⁻¹, 1303 cm⁻¹, 1361 cm⁻¹, 1372 cm⁻¹, 1433 cm⁻¹, 1453 cm⁻¹, 1491 cm⁻¹, 1547 cm⁻¹, 1557 cm⁻¹, 1576 cm⁻¹, 1610 cm⁻¹, 1660 cm⁻¹, 2868 cm⁻¹, 2930 cm⁻¹, 3059 cm⁻¹, and 3113 cm⁻¹±2 cm⁻¹; and/or
(c) has an ATR-FTIR spectrum comprising absorption bands at wavenumbers of about 677 cm⁻¹, 716 cm⁻¹, 745 cm⁻¹, 771 cm⁻¹, 784 cm⁻¹, 801 cm⁻¹, 839 cm⁻¹, 868 cm⁻¹, 906 cm⁻¹, 927 cm⁻¹, 967 cm⁻¹, 1014 cm⁻¹, 1043 cm⁻¹, 1077 cm⁻¹, 1125 cm⁻¹, 1171 cm⁻¹, 1213 cm⁻¹, 1243 cm⁻¹, 1283 cm⁻¹, 1301 cm⁻¹, 1367 cm⁻¹, 1399 cm⁻¹, 1431 cm⁻¹, 1456 cm⁻¹, 1471 cm⁻¹, 1533 cm⁻¹, 1590 cm⁻¹, 1638 cm⁻¹, 1658 cm⁻¹, 1694 cm⁻¹, 2862 cm⁻¹, 2925 cm⁻¹, 3029 cm⁻¹, and 3212 cm⁻¹±2 cm⁻¹.

6. The crystalline form of claim 5, which:
(a) has an X-Ray powder diffraction (XRPD) pattern substantially the same as shown in FIG. 14; and/or
(b) has an FT Raman spectrum substantially the same as shown in FIG. 15; and/or
(c) has an ATR-FTIR spectrum substantially the same as shown in FIG. 16.

7. The mono hydrochloric acid salt according to claim 1, which is crystalline and has an X-ray powder diffraction (XRPD) pattern comprising peaks at 5.16, 7.94, 9.98, 10.46, 10.78, 11.66, 17.28, and 18.90 [° 2 Theta±0.2° 2 Theta, Cu Kα radiation (1.5406 Å)].

8. The crystalline mono hydrochloric acid salt according to claim 7, which:
(a) has an X-ray powder diffraction (XRPD) pattern comprising peaks at 5.16, 7.94, 9.98, 10.46, 10.78, 11.66, 15.52, 15.70, 17.28, and 18.90 [° 2 Theta±0.2° 2 Theta, Cu Kα radiation (1.5406 Å)]; and/or
(b) has an FT Raman spectrum comprising absorption bands at wavenumbers of about 103 cm⁻¹, 1390 cm⁻¹, 2246 cm⁻¹, and 2930 cm⁻¹±2 cm⁻¹; and/or
(c) has an ATR-FTIR spectrum comprising absorption bands at wavenumbers of about 752 cm⁻¹, 1386 cm⁻¹, 1534 cm⁻¹, and 1602 cm⁻¹±2 cm⁻¹.

9. The crystalline mono hydrochloric acid salt according to claim 8, which:
(a) has an X-ray powder diffraction (XRPD) pattern comprising peaks at 5.16, 6.54, 7.94, 9.98, 10.46, 10.78, 11.54, 11.66, 11.98, 12.10, 13.02, 13.16, 14.38, 14.82, 15.04, 15.52, 15.70, 15.94, 16.74, 17.28, 17.74, 18.10, 18.32, 18.78, 18.90, 19.72, 20.02, 20.28, 20.80, 20.96, 21.16, 21.56, 21.62, 22.38, 22.66, 23.28, 23.50, 23.98, 24.72, 24.92, 25.20, 25.60, 25.98, 26.22, 26.98, 27.20, 27.42, 27.88, 28.10, 28.48, 28.66, 29.00, 29.70, and 30.00 [° 2 Theta±0.2° 2 Theta, Cu Kα radiation (1.5406 Å)]; and/or
(b) has an FT Raman spectrum comprising absorption bands at wavenumbers of about 103 cm⁻¹, 137 cm⁻¹, 225 cm⁻¹, 279 cm⁻¹, 330 cm⁻¹, 382 cm⁻¹, 416 cm⁻¹, 449 cm⁻¹, 479 cm⁻¹, 517 cm⁻¹, 547 cm⁻¹, 563 cm⁻¹, 596 cm⁻¹, 624 cm⁻¹, 639 cm⁻¹, 685 cm⁻¹, 758 cm⁻¹, 790 cm⁻¹, 807 cm⁻¹, 848 cm⁻¹, 878 cm⁻¹, 898 cm⁻¹, 955 cm⁻¹, 1011 cm⁻¹, 1059 cm⁻¹, 1074 cm⁻¹, 1140 cm⁻¹, 1183 cm⁻¹, 1203 cm⁻¹, 1247 cm⁻¹, 1288 cm⁻¹, 1316 cm⁻¹, 1334 cm⁻¹, 1358 cm⁻¹, 1391 cm⁻¹, 1425 cm⁻¹, 1435 cm⁻¹, 1451 cm⁻¹, 1489 cm⁻¹, 1557 cm⁻¹, 1580 cm⁻¹, 1605 cm⁻¹, 1666 cm⁻¹, 2246 cm⁻¹, 2859 cm⁻¹, 2930 cm⁻¹, 2985 cm⁻¹, 3063 cm⁻¹, and 3116 cm⁻¹±2 cm⁻¹; and/or
(c) has an ATR-FTIR spectrum comprising absorption bands at wavenumbers of about 659 cm⁻¹, 717 cm⁻¹, 731 cm⁻¹, 752 cm⁻¹, 778 cm⁻¹, 786 cm⁻¹, 803 cm⁻¹, 847 cm⁻¹, 874 cm⁻¹, 887 cm⁻¹, 906 cm⁻¹, 931 cm⁻¹, 950 cm⁻¹, 971 cm⁻¹, 1010 cm⁻¹, 1017 cm⁻¹, 1080 cm⁻¹, 1098 cm⁻¹, 1134 cm⁻¹, 1148 cm⁻¹, 1180 cm⁻¹, 1215 cm⁻¹, 1248 cm⁻¹, 1282 cm⁻¹, 1334 cm⁻¹, 1385 cm⁻¹, 1439 cm⁻¹, 1459 cm⁻¹, 1534 cm⁻¹, 1602 cm⁻¹, 1659 cm⁻¹, 1682 cm⁻¹, 2936 cm⁻¹, 3054 cm⁻¹, 3239 cm⁻¹, and 3434 cm⁻¹±2 cm⁻¹.

10. The crystalline mono hydrochloric acid salt according to claim 9, which:
(a) has an X-Ray powder diffraction (XRPD) pattern substantially the same as shown in FIG. 1; and/or
(b) has an FT Raman spectrum substantially the same as shown in FIG. 2; and/or
(c) has an ATR-FTIR spectrum substantially the same as shown in FIG. 3.

11. The crystalline mono hydrochloric acid salt according to claim 7, which:
(a) has an X-ray powder diffraction (XRPD) pattern comprising peaks at 5.20, 7.94, 9.92, 10.36, 10.68, 11.60, 15.58, 17.16, and 18.78 [° 2 Theta±0.2° 2 Theta, Cu Kα radiation (1.5406 Å)]; and/or
(b) has an FT Raman spectrum comprising absorption bands at wavenumbers of about 163 cm⁻¹, 1391 cm⁻¹, 2920 cm⁻¹, and 2948 cm⁻¹±2 cm⁻¹; and/or
(c) has an ATR-FTIR spectrum comprising absorption bands at wavenumbers of about 753 cm⁻¹, 1387 cm⁻¹, 1541 cm⁻¹, 1604 cm⁻¹±2 cm⁻¹.

12. The crystalline mono hydrochloric acid salt according to claim 11, which:
(a) has an X-ray powder diffraction (XRPD) pattern comprising peaks at 5.20, 6.52, 7.94, 9.92, 10.36, 10.68, 11.60, 12.04, 13.02, 13.10, 14.34, 14.70, 14.92, 15.58, 15.94, 16.68, 17.16, 17.52, 17.82, 18.06, 18.22, 18.78, 19.70, 19.80, 19.96, 20.64, 20.88, 21.10, 21.50, 22.30, 22.58, 23.18, 23.44, 23.58, 23.94, 24.22, 24.70, 25.02, 25.42, 25.74, 26.18, 26.82, 27.04, 27.24, 27.72, 27.90, 28.34, 28.86, 29.10, 29.52, 29.74, 30.14, 31.08, and 31.52 [° 2 Theta±0.2° 2 Theta, Cu Kα radiation (1.5406 Å)]; and/or
(b) has an FT Raman spectrum comprising absorption bands at wavenumbers of about 107 cm⁻¹, 135 cm⁻¹, 163 cm⁻¹, 225 cm⁻¹, 336 cm⁻¹, 417 cm⁻¹, 450 cm⁻¹, 480 cm⁻¹, 518 cm⁻¹, 546 cm⁻¹, 562 cm⁻¹, 596 cm⁻¹, 621 cm⁻¹, 639 cm⁻¹, 686 cm⁻¹, 758 cm⁻¹, 774 cm⁻¹, 790 cm⁻¹, 813 cm⁻¹, 848 cm⁻¹, 877 cm⁻¹, 899 cm⁻¹, 955 cm⁻¹, 1011 cm⁻¹, 1046 cm⁻¹, 1059 cm⁻¹, 1075 cm⁻¹, 1140 cm⁻¹, 1188 cm⁻¹, 1200 cm⁻¹, 1234 cm⁻¹, 1249 cm⁻¹, 1289 cm⁻¹, 1317 cm⁻¹, 1335 cm⁻¹, 1358 cm⁻¹, 1391 cm⁻¹, 1426 cm⁻¹, 1435 cm⁻¹, 1452 cm⁻¹, 1489 cm⁻¹, 1558 cm⁻¹, 1578 cm⁻¹, 1606 cm⁻¹, 1660 cm⁻¹, 2920 cm⁻¹, 2948 cm⁻¹, 2986 cm⁻¹, and 3059 cm⁻¹±2 cm⁻¹; and/or
(c) has an ATR-FTIR spectrum comprising absorption bands at wavenumbers of about 673 cm⁻¹, 718 cm⁻¹, 739 cm⁻¹, 753 cm⁻¹, 773 cm⁻¹, 779 cm⁻¹, 803 cm⁻¹, 848 cm⁻¹, 875 cm⁻¹, 888 cm⁻¹, 898 cm⁻¹, 906 cm⁻¹, 932 cm⁻¹, 946 cm⁻¹, 971 cm⁻¹, 1010 cm⁻¹, 1017 cm⁻¹, 1037 cm⁻¹, 1067 cm⁻¹, 1082 cm⁻¹, 1095 cm⁻¹, 1134 cm⁻¹, 1149 cm⁻¹, 1169 cm⁻¹, 1181 cm⁻¹, 1223 cm⁻¹, 1248 cm⁻¹, 1286 cm⁻¹, 1333 cm⁻¹, 1387 cm⁻¹, 1406 cm⁻¹, 1424 cm⁻¹, 1440 cm⁻¹, 1459 cm⁻¹, 1467 cm⁻¹, 1541 cm⁻¹, 1585 cm⁻¹, 1604 cm⁻¹, 1658 cm⁻¹, 2937 cm⁻¹, 3054 cm⁻¹, 3230 cm⁻¹, 3349 cm⁻¹, and 3429 cm⁻¹±2 cm⁻¹.

13. The crystalline mono hydrochloric acid salt according to claim 12, which:
(a) has an X-Ray powder diffraction (XRPD) pattern substantially the same as shown in FIG. 4; and/or
(b) has an FT Raman spectrum substantially the same as shown in FIG. 5; and/or
(c) has an ATR-FTIR spectrum substantially the same as shown in FIG. 6.

14. The crystalline mono hydrochloric acid salt according to claim 7, which has an X-ray powder diffraction (XRPD) pattern comprising peaks at 5.6, 7.14, 8.66, 9.92, 10.52, 10.88, 11.92, 15.6, 16.64, 17.7, and 20.94 [° 2 Theta±0.2° 2 Theta, Cu Kα radiation (1.5406 Å)].

15. The crystalline mono hydrochloric acid salt according to claim 14, which has an X-ray powder diffraction (XRPD) pattern substantially the same as shown in FIG. 7.

16. The crystalline mono hydrochloric acid salt according to claim 7, which has an X-ray powder diffraction (XRPD) pattern comprising peaks at 5.22, 8.16, 9.88, 10.04, 10.48, 10.8, 11.48, 11.72, 12.2, 12.58, 13.1, 13.62, 14.5, 14.82, 15.12, 15.7, 16.02, 17.02, 17.82, 18.16, 18.42, 18.58, 18.72, 18.9, 19.4, 19.62, and 19.84 [° 2 Theta±0.2° 2 Theta, Cu Kα radiation (1.5406 Å)].

17. The crystalline mono hydrochloric acid salt according to claim 14, which has an X-ray powder diffraction (XRPD) pattern substantially the same as shown in FIG. 8.

18. The mono hydrochloric acid salt according to claim 1, which is amorphous and
(a) has an FT Raman spectrum comprising absorption bands at wavenumbers of about 1296 cm⁻¹, 1608 cm⁻¹, 2928 cm⁻¹, and 3060 cm⁻¹±2 cm⁻¹±2 cm⁻¹; and/or
(b) has an ATR-FTIR spectrum comprising absorption bands at wavenumbers of about 742 cm⁻¹, 778 cm⁻¹, 1377 cm⁻¹, and 1531 cm⁻¹±2 cm⁻¹.

19. The amorphous mono hydrochloric acid salt according to claim 18, which
(a) has an FT Raman spectrum comprising absorption bands at wavenumbers of about 334 cm⁻¹, 415 cm⁻¹, 543 cm⁻¹, 641 cm⁻¹, 683 cm⁻¹, 759 cm⁻¹, 803 cm⁻¹, 842 cm⁻¹, 879 cm⁻¹, 912 cm⁻¹, 1011 cm⁻¹, 1061 cm⁻¹, 1077 cm⁻¹, 1137 cm⁻¹, 1203 cm⁻¹, 1296 cm⁻¹, 1359 cm⁻¹, 1381 cm⁻¹, 1437 cm⁻¹, 1453 cm⁻¹, 1557 cm⁻¹, 1579 cm⁻¹, 1609 cm⁻¹, 2928 cm⁻¹, and 3059 cm⁻¹±2 cm⁻¹; and/or
(b) has an ATR-FTIR spectrum comprising absorption bands at wavenumbers of about 680 cm⁻¹, 716 cm⁻¹, 742 cm⁻¹, 778 cm⁻¹, 841 cm⁻¹, 868 cm⁻¹, 1010 cm⁻¹, 1043 cm⁻¹, 1078 cm⁻¹, 1129 cm⁻¹, 1177 cm⁻¹, 1224 cm⁻¹, 1285 cm⁻¹, 1376 cm⁻¹, 1403 cm⁻¹, 1456 cm⁻¹, 1531 cm⁻¹, 1582 cm⁻¹, 1627 cm⁻¹, 2924 cm⁻¹, and 3243 cm⁻¹±2 cm⁻¹.

20. The amorphous mono hydrochloric acid salt according to claim 19, which:
(a) has an FT Raman spectrum substantially the same as shown in FIG. 9; and/or
(b) has an ATR-FTIR spectrum substantially the same as shown in FIG. 10.

21. The crystalline form of claim 3, which:
(a) has an X-ray powder diffraction (XRPD) pattern comprising peaks at 5.46, 8.08, 10.36, 11.56, 11.86, 16.56 and 17.44 [° 2 Theta±0.2° 2 Theta, Cu Kα radiation (1.5406 Å)]; and/or
(b) has an FT Raman spectrum comprising absorption bands at wavenumbers of about 838 cm⁻¹, 1079 cm⁻¹, 1373 cm⁻¹, and 1608 cm⁻¹±2 cm⁻¹; and/or
(c) has an ATR-FTIR spectrum comprising absorption bands at wavenumbers of about 746 cm⁻¹, 784 cm⁻¹, 1371 cm⁻¹, and 3437 cm⁻¹±2 cm⁻¹.

22. The crystalline form of claim 21, which:
(a) has an X-ray powder diffraction (XRPD) pattern comprising peaks at 5.46, 8.08, 9.90, 10.36, 11.56, 11.86, 12.46, 12.84, 13.36, 13.70, 13.86, 14.82, 14.98, 15.28, 16.56, 17.00, 17.44, 17.72, 18.18, 18.42, 18.64, 18.94, 19.26, 19.80, 20.12, 20.52, 20.78, 21.02, 21.14, 21.34, 21.48, 21.66, 21.80, 22.74, 22.84, 23.22, 23.42, 23.72, 24.02, 24.42, 24.58, 25.10, 25.52, 25.92, 26.18, 26.96, 27.60, 27.82, 27.94, 28.64, 29.16, and 29.92 [° 2 Theta±0.2° 2 Theta, Cu Kα radiation (1.5406 Å)]; and/or
(b) has an FT Raman spectrum comprising absorption bands at wavenumbers of about 233 cm⁻¹, 334 cm⁻¹, 415 cm⁻¹, 490 cm⁻¹, 540 cm⁻¹, 575 cm⁻¹, 596 cm⁻¹, 625 cm⁻¹, 642 cm⁻¹, 680 cm⁻¹, 699 cm⁻¹, 719 cm⁻¹, 757 cm⁻¹, 791 cm⁻¹, 810 cm⁻¹, 838 cm⁻¹, 879 cm⁻¹, 899 cm⁻¹, 1010 cm⁻¹, 1060 cm⁻¹, 1080 cm⁻¹, 1134 cm⁻¹, 1204 cm⁻¹, 1234 cm⁻¹, 1288 cm⁻¹, 1345 cm⁻¹, 1359 cm⁻¹, 1374 cm⁻¹, 1437 cm⁻¹, 1556 cm⁻¹, 1576 cm⁻¹, 1608 cm⁻¹, 2867 cm⁻¹, 2928 cm⁻¹, 3062 cm⁻¹, and 3114 cm⁻¹±2 cm⁻¹; and/or
(c) has an ATR-FTIR spectrum comprising absorption bands at wavenumbers of about 676 cm⁻¹, 716 cm⁻¹, 746 cm⁻¹, 774 cm⁻¹, 784 cm⁻¹, 838 cm⁻¹, 868 cm⁻¹, 904 cm⁻¹, 933 cm⁻¹, 1009 cm⁻¹, 1043 cm⁻¹, 1077 cm⁻¹, 1125 cm⁻¹, 1175 cm⁻¹, 1216 cm⁻¹, 1249 cm⁻¹, 1286 cm⁻¹, 1301 cm⁻¹, 1370 cm⁻¹, 1457 cm⁻¹, 1470 cm⁻¹, 1533 cm⁻¹, 1587 cm⁻¹, 1651 cm⁻¹, 2863 cm⁻¹, 2927 cm⁻¹, 3041 cm⁻¹, 3223 cm⁻¹, and 3437 cm⁻¹±2 cm⁻¹.

23. The crystalline form of claim 22, which:
(a) has an X-Ray powder diffraction (XRPD) pattern substantially the same as shown in FIG. 11; and/or
(b) has an FT Raman spectrum substantially the same as shown in FIG. 12; and/or
(c) has an ATR-FTIR spectrum substantially the same as shown in FIG. 13.

24. The zwitterion according to claim 2, which is amorphous.

25. The amorphous compound according to claim 24, which:
(a) has an FT Raman spectrum comprising absorption bands at wavenumbers of about 1296 cm⁻¹, 1608 cm⁻¹, 2928 cm⁻¹, and 3060 cm⁻¹±2 cm⁻¹±2 cm⁻¹; and/or
(b) has an ATR-FTIR spectrum comprising absorption bands at wavenumbers of about 742 cm⁻¹, 778 cm⁻¹, 1379 cm⁻¹, and 1536 cm⁻¹±2 cm⁻¹.

26. The amorphous compound according to claim 25, which
(a) has an FT Raman spectrum comprising absorption bands at wavenumbers of about 335 cm⁻¹, 415 cm⁻¹, 544 cm⁻¹, 641 cm⁻¹, 684 cm⁻¹, 759 cm⁻¹, 801 cm⁻¹, 845 cm⁻¹, 879 cm⁻¹, 910 cm⁻¹, 1011 cm⁻¹, 1061 cm⁻¹, 1077 cm⁻¹, 1137 cm⁻¹, 1203 cm⁻¹, 1296 cm⁻¹, 1359 cm⁻¹, 1386 cm⁻¹, 1438 cm⁻¹, 1556 cm⁻¹, 1579 cm⁻¹, 1608 cm⁻¹, 2862 cm⁻¹, 2927 cm⁻¹, and 3060 cm⁻¹±2 cm⁻¹; and/or
(b) has an ATR-FTIR spectrum comprising absorption bands at wavenumbers of about 679 cm⁻¹, 716 cm⁻¹, 742 cm⁻¹, 778 cm⁻¹, 842 cm⁻¹, 869 cm⁻¹, 928 cm⁻¹, 1010 cm⁻¹, 1043 cm⁻¹, 1078 cm⁻¹, 1127 cm⁻¹, 1182 cm⁻¹, 1224 cm⁻¹, 1285 cm⁻¹, 1379 cm⁻¹, 1455 cm⁻¹, 1537 cm⁻¹, 1589 cm⁻¹, 1632 cm⁻¹, 2863 cm⁻¹, 2926 cm⁻¹, 3047 cm⁻¹, and 3255 cm⁻¹±2 cm⁻¹.

27. The amorphous compound according to claim 26, which:
(a) has an FT Raman spectrum substantially the same as shown in FIG. 9; and/or
(b) has an ATR-FTIR spectrum substantially the same as shown in FIG. 17.

28. The crystalline form of claim 3, which has an X-ray powder diffraction (XRPD) pattern comprising peaks at 6.93, 7.28, 12.11, 14.52, 15.04, 15.73, 19.44, and 22.00 [° 2 Theta±0.2° 2 Theta, Cu Kα radiation (1.5406 Å)].

29. The crystalline form of claim 28, which has an X-ray powder diffraction (XRPD) pattern comprising peaks at 6.93, 7.28, 8.07, 10.08, 12.11, 13.53, 13.92, 14.52, 15.04, 15.73, 16.21, 16.96, 17.57, 18.13, 18.46, 19.31, 19.44, 19.87, 20.25, 20.51, 20.97, 22.00, 22.29, 22.49, 22.93, 23.12, 23.33, 23.60, 24.03, 24.40, 24.92, 25.04, 25.26, 25.62, 25.86, 26.15, 26.52, 27.26, 28.66, and 30.58 [° 2 Theta±0.2° 2 Theta, Cu Kα radiation (1.5406 Å)].

30. The crystalline form of claim 28, which has an X-ray powder diffraction (XRPD) pattern substantially the same as shown in FIG. 18.

31. A pharmaceutical composition comprising a compound according to claim 3, and at least one additional ingredient selected from pharmaceutically acceptable carriers, diluents and excipients.

32. The pharmaceutical composition according to claim 31, wherein the pharmaceutical composition is in a form suitable for intravenous administration to a mammal.

33. A solution for intravenous administration to a mammal, comprising:
(i) a compound according to claim 2;
(ii) water for injection; and
(iii) sodium chloride.

34. The solution for intravenous administration according to claim 33, wherein the concentration of the compound is 50 mg/mL.

35-36. (canceled)

37. A method of treating bacterial infections and resulting diseases in a mammal, said method comprising administering a therapeutically effective amount of a compound according to claim 3 to said mammal.

38-40. (canceled)

41. A pharmaceutical composition comprising a compound according to claim 7, and at least one additional ingredient selected from pharmaceutically acceptable carriers, diluents and excipients.

42. A pharmaceutical composition comprising a compound according to claim 18, and at least one additional ingredient selected from pharmaceutically acceptable carriers, diluents and excipients.

43. A pharmaceutical composition comprising a compound according to claim 24, and at least one additional ingredient selected from pharmaceutically acceptable carriers, diluents and excipients.

44. A pharmaceutical composition comprising a mixture of one or more compounds according to claims 3, 7, 18 and 24, and at least one additional ingredient selected from pharmaceutically acceptable carriers, diluents and excipients.

45. The pharmaceutical composition of claim 44, wherein the compounds are amorphous.

46. A method of treating bacterial infections and resulting diseases in a mammal, said method comprising administering to said mammal a therapeutically effective amount of a compound according to claim 7.

47. A method of treating bacterial infections and resulting diseases in a mammal, said method comprising administering to said mammal a therapeutically effective amount of a compound according to claim 18.

48. A method of treating bacterial infections and resulting diseases in a mammal, said method comprising administering to said mammal a therapeutically effective amount of a compound according to claim 24.

49. A solution for intravenous administration to a mammal, comprising:
(i) a compound according to claim 1;
(ii) water for injection; and
(iii) sodium chloride.

50. The solution for intravenous administration according to claim 49,
wherein the concentration of the compound is 50 mg/mL.
